(19) United States
(12) Patent Application Publication
(10) Pub. No.: US 2016/0279187 A1
(43) Pub. Date: Sep. 29, 2016

(54) CALPAIN INHIBITORS FOR IBD AND COLORECTAL CANCER TREATMENT

(71) Applicant: UNIVERSITY OF HAWAII, Honolulu, HI (US)

(72) Inventor: Peter HOFFMANN, Honolulu, HI (US)

(21) Appl. No.: 15/033,555

(22) PCT Filed: Oct. 29, 2014
(86) PCT No.: PCT/US14/62960

§ 371 Date: Apr. 29, 2016

Related U.S. Application Data
(60) Provisional application No. 62/013,357, filed on Jun. 17, 2014.
(60) Provisional application No. 61/898,031, filed on Oct. 31, 2013.

(51) Int. Cl. A61K 38/06; A61K 9/51; A61K 45/06

(52) U.S. Cl. CPC A61K 38/06; A61K 45/06; A61K 9/5161; A61K 9/5153

(57) ABSTRACT

The present disclosure provides a method of treating colitis and colorectal cancer with a m-calpain selective inhibitor. Also described in this disclosure are pharmaceutical compositions comprising a m-calpain selective inhibitor to inhibit m-calpain activity in tumor cells and other cells in the colon.

### GOVERNMENT RIGHTS

This invention was made with government support under RO1 AI08999 awarded by National Institute of Health and National Institute of Allergy and Infectious Diseases. The government has certain rights in the invention.

### FIELD OF THE INVENTION

This invention relates to methods of treating inflammatory bowel disease and colorectal cancer. The invention further relates to methods of treating inflammation in the colon, and includes kits comprising compositions for use in the methods of this invention.

### BACKGROUND

Chronic inflammation drives the pathogenesis of several health disorders, including inflammatory bowel disease (IBD), Crohn's disease and ulcerative colitis. A long-term complication of chronic inflammation associated with IBD is the development of colorectal cancer, which is one of the leading causes of cancer-related death in the western hemisphere (Clevers H. Cell; 118: 671-674, 2004). The cumulative risk for acquiring colorectal cancer can increase to approximately 20% in patients with IBD who live for 30 years with the disease (Eaden J. A., Abrams K. R., Mayberry J. F., Gut; 48: 526-535, 2001; Sachar D. B., Gut; 35: 1507-1508, 1994). Moreover, clinical studies have shown that patients with colitis have a 2- to 8-fold relative risk of developing colorectal cancer compared to the general population (Ullman T. A., Itzkowitz S. H., Gastroenterology, 140: 1807-1816, 2011).

Calpain enzymes are upregulated in IBD tissues as well as chronically inflamed colon tissue (Huang Z., Rose A. H., Hoffmann F. W., et al. J Immunol, 191: 3778-3788, 2013). Calpains are Ca²⁺-activated cysteine proteases that cleave specific targets to modulate cellular functions relevant to inflammation such as proliferation, migration, and apoptosis (Saido T C, Sorimachi H, Suzuki K., FASEB journal: official publication of the Federation of American Societies for Experimental Biology; 8: 814-822, 1994). The two major isoforms of this enzyme, calpain-1 (or μ-calpain) and calpain-2 (or m-calpain), require micomolar and millimolar Ca²⁺ concentrations for activity, respectively (Brown N., Crawford C., FEBS letters, 322: 65-68, 1993). Following activation by intracellular Ca²⁺, calpains cleave a specific subset of cellular proteins, including cytoskeletal proteins and proteins involved in signal transduction (duVerle D, Takigawa I, Ono Y, et al., Genome informatics International Conference on Genome Informatics, 22: 202-213, 2010). In macrophages, calpain-2 is the predominant isoform.

### SUMMARY OF THE INVENTION

In one aspect, this invention features treating colorectal cancer or inflammatory bowel disease with a selective m-calpain inhibitor, or a pharmaceutically acceptable salt, hydrate, or solute thereof. Inflammatory bowel disease may refer, for example, to any of the following diseases, syndromes or conditions: ulcerative colitis, Crohn's disease, collagenous colitis, lymphocytic colitis, ischaemic colitis, diversion colitis, Behçet's syndrome, infective colitis or indeterminate colitis.

In some aspects, the method of treatment can further comprise administering a therapeutically effective amount of an agent useful for treating inflammatory bowel disease. The m-calpain selective inhibitor, or a pharmaceutically acceptable salt, hydrate, or solute thereof, may be co-administered with a therapeutically effective amount of an agent useful for treating inflammatory bowel disease or colorectal cancer either sequentially or simultaneously. The m-calpain selective inhibitor, or a pharmaceutically acceptable salt, hydrate, or solute thereof, and the therapeutically effective amount of an agent useful for treating inflammatory bowel disease or colorectal cancer can be administered within about one hour of each other, within about one day or each other, or within about one week of each other, or within about one month of each other. In some embodiments, inflammation of the colon can be reduced by administering to a subject in need thereof, a therapeutically effective amount of a pharmaceutical composition comprising a m-calpain selective inhibitor, or a pharmaceutically acceptable salt, hydrate, or solute thereof.

In some aspects, the m-calpain selective inhibitor can be co-administered together with or separately from a therapeutic agent useful for treating inflammatory bowel disease and/or reducing inflammation of the colon. In some aspects, the therapeutic agent useful for treating inflammatory bowel disease can be selected from one of the following classes of compounds: 5-aminosalicyclic acids, corticosteroids, thiopurines, tumor necrosis factor-α blockers and JAK inhibitors.

In some aspects, the m-calpain selective inhibitor can be co-administered together with or separately from a therapeutic agent useful for treating treating colorectal cancer. In some aspects, the therapeutic agent useful for treating colorectal cancer can be selected from one of the following classes of compounds: antineoplastic agents, thymidylate synthase inhibitors, topiosomerase inhibitors, multi-kinase inhibitors, endothelial growth factor receptor (EGFR) inhibitors, vascular endothelial growth factor receptor (VEGF) inhibitors, NFkB inhibitors, angiogenesis inhibitors, anti-metabolites, and anti-cytokine inhibitors. In some aspects, the methods featured herein for treating colorectal cancer can be characterized by the inhibition or reduction of cancer progression. In some aspects, the methods featured herein for treating colorectal cancer can be characterized by the inhibition or reduction of inflammatory bowel disease. Thus, in some aspects, this invention features inhibiting the growth of tumor cells or a colony of tumor cells by contacting said tumor cells with an effective amount of an selective inhibitor of m-calpain. In some aspects, the tumor cells can comprise colorectal cancer cells.

The level of m-calpain activity can be reduced in colorectal cancer cells or other cells in the colon by administering to a subject a therapeutically effective amount of a pharmaceutical composition comprising a m-calpain selective inhibitor, or a pharmaceutically acceptable salt, hydrate, or solute thereof. In some aspects, the m-calpain selective inhibitor can be co-administered together with or separately from a therapeutic agent useful for treating treating colorectal cancer.

The therapeutically effective dose of the m-calpain specific inhibitor for use in the methods featured herein may range from about 0.01 mg/kg to about 1 mg/kg, from about 0.02 mg/kg to about 50 mg/kg, from about 0.1 mg/kg to 50 mg/kg, from about 0.1 mg/kg to 5 mg/kg, from about 0.1 mg/kg to 2 mg/kg, or from about 0.1 mg/kg to 1 mg/kg. The therapeutically effective dose may be, for example, about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, 30, 35, 40, 45 or about 50 mg/kg, or any range in between any two of the recited doses. In some aspects, the dose will be 0.08 mg/kg to about 0.5 mg/kg, from about 0.08 to about 0.24 mg/kg, or from about 0.24 to about 0.85 mg/kg. In one aspect, the effective dose of the m-calpain inhibitor can be administered in one or more doses, with the therapeutically effective dose of, for example, 0.08, 0.24, 0.5, 0.75, 1.0, or 1.25 mg/kg for each dose. In one embodiment, the dose is administered by a delivery route selected from the group consisting of intraperitoneal, intradermal, intramuscular, intraperitoneal, intravenous, topical, subcutaneous, anal, or epidural routes. In one embodiment, the one or more effective doses of the m-calpain inhibitor can be administered orally, intravenously, intramuscularly, or subcutaneously. In one embodiment, the one or more effective doses of the m-calpain inhibitor can be administered orally. In one aspect, the one or more effective doses of the m-calpain inhibitor can be administered intravenously. In certain embodiments, the one or more effective doses of the m-calpain inhibitor can be administered subcutaneously. In one embodiment, the one or more effective doses of the m-calpain inhibitor can be administered anally.

In some aspects, the m-calpain selective inhibitor can be administered as a pharmaceutical composition. In some aspects the pharmaceutical composition may be, for example, an immediate release formulation or a controlled release formulation, for example, a delayed release formulation. Various delivery systems can be used to administer a selective m-calpain inhibitor in accordance with the methods of the invention, e.g., immediate or controlled release delivery systems, for example, delayed release delivery systems. In some embodiments, the delivery system can include, for example, encapsulation in liposomes, particles or microcapsules. In some aspects the pharmaceutical composition can comprise a formulation further comprising one or a plurality of particles. In some aspects the particle composition is suitable for use in delayed release administration.

A delayed release administration method allows for colon-targeted drug delivery, which reduces the therapeutically effective dose required compared to systemic dosages. In some aspects, the therapeutically effective amount of the m-calpain specific inhibitor when administered in a particle formulation can be from about 0.1 ng/kg to 4.0 mg/kg, from about 0.5 ng/kg to about 0.5 mg/kg, from about 1.0 ng to about 100 ug/kg, from about 10 ng/kg to about 10 ug/kg, from about 100 ng/kg to about 1 ug/kg, from about 1 mg/kg to about 100 mg/kg, or from about 10 mg/kg to about 1000 mg/kg.

In some aspects, the m-calpain selective inhibitor (or “selective m-calpain inhibitor”, or “selective calpain-2 inhibitor”) can be a modified peptide that comprises at least one partial leucine moiety and an electrophilic moiety. In some aspects, the electrophilic moiety is an alkyl halide group. In some aspects, the m-calpain selective inhibitor is ZZ-LLY-CH2F (“Calpain Inhibitor IV”, or “ZZ-LLY-FMK”, or “z-LLY-CH2F”, or “zLLY-CH2F”, or “zz-LLY-CH2F”, or “zzLLY-CH2F”, or “zLLY-CH2F”).

In some aspects, the m-calpain selective inhibitor can be an irreversible inhibitor of m-calpain. In some aspects, the m-calpain selective inhibitor can irreversibly inhibit m-calpain with a k₂ rate constant greater than about 28,000 M⁻¹s⁻¹, or greater than 25,000 M⁻¹s⁻¹, or greater than 20,000 M⁻¹s₋₁, or greater than 15,000 M⁻¹s⁻¹, or greater than 10,000 M⁻¹s⁻¹ or greater than 5,000 M⁻¹s⁻¹.

In some aspects, the ratio of inhibition rates k₂ of the on-target m-calpain to an off-target protein can be greater than about 0.5, or greater than 0.1, or greater than 0.05, or greater than 0.01. In some aspects the off-target protein is μ calpain.

In some aspects, the m-calpain selective inhibitor can be a reversible inhibitor of m-calpain. In some aspects, the m-calpain selective inhibitor selectivity can be defined as inhibiting or binding to m-calpain significantly more than μ-calpain. The ratio of the IC50 of the m-calpain selective inhibitor to the off-target μ-calpain compared to the IC50 of the m-calpain selective inhibitor to the on-target m-calpain can be greater than 10, greater than 20, or greater than 100.

In some aspects, the m-calpain selective inhibitor can be a reversible inhibitor of m-calpain. In some aspects, the m-calpain selective inhibitor can reversibly inhibit m-calpain with a IC₅₀ of less than about 50 nM, or less than 40 nM, or less than 30 nM, or less than 20 nM, or less than 10 nM, or less than 1 nM. In some aspects, the m-calpain selective inhibitor selectivity can be defined as inhibiting or binding to m-calpain more than to μ-calpain, as described herein.

This disclosure also describes a kit, comprising a first composition comprising an effective amount of a selective m-calpain inhibitor wherein the selective m-calpain inhibitor is Calpain Inhibitor IV, or a pharmaceutically acceptable salt, hydrate, or solute thereof; and a second composition a therapeutic agent useful for treating inflammatory bowel disease or colorectal cancer, or a pharmaceutically acceptable salt, hydrate, or solute thereof, together with instructions for administering the first composition and the second composition to a patient suffering from colitis. In some aspects, the first and second composition of the kit can be administered in combination, can be administered simultaneously, can be administered separately, can be administered sequentially, or can be administered in a controlled manner.

### BRIEF DESCRIPTION OF THE DRAWINGS

FIG. 1 shows a Calpain-2 inhibitor treatment reduces colitis. Panel A shows a dose-response experiment conducted in which mice were treated with 2% DSS for 4 d and interperitonally injected daily with a calpain-2 inhibitor (zLLY-CH2F) at concentrations ranging from 0 to 1.25 mg/kg. Panel B shows a chronic AOM/DSS protocol modified to include intervention on day 8 with daily interperitonal injections of either vehicle control or 0.75 mg/kg calpain-2 inhibitor. Panel C shows the percent of initial body weight for a cohort of mice over timed exposure, wherein mice were weighed every other day for the 63 d protocol (n=20/group). Data represent mean±SEM and weight curves were analyzed by a repeated measures one-way ANOVA (F2,31=82.02; P<0.0001) followed by Tukey post-test. Panel D shows the results of analysis of hemoglobin in Cecum for the cohorts wherein bloody diarrhea was assessed on day 63 using an ELISA to measure hemoglobin in cecums. Groups were analyzed using a one-way ANOVA followed by Tukey post-test. Data represent mean±SEM.

FIG. 2 shows Calpain-2 inhibitor treatment reduces pathology and inflammation in colons. Panel A shows paraffin embedded sections H&E stained, with 20× representative images from each group shown (scalebar=100 μm). Panel B shows blind scores for colitis by assessment of 5 sections from each colon. Panels C and D show real-time PCR results of total RNA extracted from colons to assess expression of cytokine mRNAs. Groups were analyzed using one way Anova followed by Tukey post-test. Data represent mean+SEM.

FIG. 3 shows Calpain-2 inhibition in bone marrow derived macrophages reduces inflammatory cytokine expression. Panel A shows cytokine mRNA expression levels of IFNγ, IL-6, TNFα, MCP-1, IL-10, and IL-12p70 in bone marrow derived macrophages. Panel B shows cytokine mRNA expression levels for NFκB and IκB.

FIG. 4 shows Calpain-2 inhibitor treatment results in smaller colorectal tumor volumes and reduced cancer pathology. Panel A shows in mice subjected to the AOM/DSS model of colitis large colon tumors visible in the distal regions near the rectum, and significant reduction of this tumor volume after calpain-2 inhibitor treatment. Panel B shows the measurements of colon tumors from mice (n=20), a statistically significant reduction in tumor volume p<0.0001 by Tukey post-test. Data represent mean±SEM. Panel C shows colon cancer pathology evident in AOM/DSS mice and calpain-2 inhibitor treated mice were significantly alleviated in the pathology. Panel D shows blind colon cancer scores by an independent Pathologist for the various cohorts. Data represent mean+SEM (n=20) and means were compared by one way ANOVA followed by Tukey post-test, p<0.05.

FIG. 5 shows Calpain-2 inhibition in mouse and human colon cancer cells slows proliferation of the cells by inhibition of calpain degradation of IκB. Panel A shows CT-26 WT mouse colon cancer cells and HT-29 human colorectal cancer cells treated with either calpain-2 inhibitor or DMSO over time. Panel B shows calpain activity inhibition in the cell lines as determined using a Calpain Activity Assay Kit (BioVision). Panel C shows western blots of cell lysate for IκB blots and on density centrifuged nuclear samples for NFκB blots. Panel D shows densitometry analysis of the blots.

FIG. 6 shows that mice treated with the m-calpain inhibitor had a very significant tumor reduction of colitis-associated cancer compared to mice treated with vehicle control. Panel A shows the images of the tumors for two of the mice treated. Panel B shows the tumor volume data of the cohorts. Data represent mean+SEM (n=20) and means were compared by one way ANOVA followed by Tukey post-test, p<0.05.

FIG. 6 shows that injections with an m-calpain inhibitor reduced tumor formation as shown by representative images in Panel A, and tumor volume data in Panel B.

FIG. 7 shows the mechanism by which treatment with a calpain-2 inhibitor, zLLY-CH2F, limits both macrophage-driven inflammation and colon cancer cell proliferation. This two-hit effect provides a potent therapeutic intervention for reducing colorectal cancer arising from chronic intestinal inflammation.

FIG. 8 shows the structure of an exemplary m-calpain containing chitosan/alginate coated nanoparticle (400 nm diameter) for targeted delivery to the colon.

FIG. 9 shows the chemical structure of Z-LLY-CH2F (“Calpain Inhibitor IV”), a selective irreversible inhibitor of m-calpain.

FIG. 10 shows the chemical structure of a class of selective irreversible inhibitors of m-calpain exhibiting an electrophilic moiety (E) at the carboxy terminus.

FIG. 11 shows the chemical structure of a class of selective irreversible inhibitors of m-calpain exhibiting an alkyl halide moiety (X) at the carboxy terminus.

### DETAILED DESCRIPTION

The present invention is based on a surprising, and unexpected, discovery that the m-calpain specific inhibitors of this invention are potent, selective inhibitors of m-calpain with anti-inflammatory and anti-tumor activity. In addition, aspects of the present invention are based on the surprising discovery that the potent and selective m-calpain inhibitors of this invention are useful in treating cancer and/or treating inflammatory bowel disease.

The instant disclosure provides methods of treating a subject by administering one or more effective dose(s) of selective m-calpain inhibitors to achieve the desired therapeutic effect, for example, treating cancer and/or treating inflammatory bowel disease. The subject is preferably a mammal, including, but not limited to, animals such as cows, pigs, horses, chickens, cats, dogs, or other domesticated animals, and is preferably human.

Various delivery systems can be used to administer a selective m-calpain inhibitor in accordance with the methods of the invention, e.g., immediate or controlled release delivery systems, for example, delayed release delivery systems. In some embodiments, the delivery system can include, for example, encapsulation in liposomes, particles or microcapsules. Methods of introduction include, but are not limited to, topical, galvage, subcutaneous, intradermal, intramuscular, intraperitoneal, intravenous, anal, subcutaneous, intranasal, epidural, and oral routes. For treatment of certain cancers, oral, topical, subcutaneous, intradermal, and systemic deliveries can be particularly efficacious.

Featured herein is a method of treating colorectal cancer and/or inflammatory bowel disease with a selective m-calpain inhibitor, or a pharmaceutically acceptable salt, hydrate, or solute thereof.

Inflammatory bowel disease may refer to any of the following diseases, syndromes or conditions: ulcerative colitis, Crohn's disease, collagenous colitis, lymphocytic colitis, ischaemic colitis, diversion colitis, Behcet's syndrome, infective colitis or indeterminate colitis.

In some aspects, the method of treatment can further comprise administering a therapeutically effective amount of an agent useful for treating inflammatory bowel disease, and/or inflammation of the colon. The m-calpain selective inhibitor, or a pharmaceutically acceptable salt, hydrate, or solute thereof, may be administered with a therapeutically effective amount of an agent useful for treating inflammatory bowel disease or colorectal cancer, either sequentially or simultaneously (“coadministration”). The m-calpain selective inhibitor, or a pharmaceutically acceptable salt, hydrate, or solute thereof, and the therapeutically effective amount of an agent useful for treating inflammatory bowel disease or colorectal cancer can be administered within about one hour of each other, within about one day or each other, within about one week of each other, or within about one month of each other.

In some aspects, the m-calpain selective inhibitor can be administered first as part of a coadministration with a therapeutic agent useful for treating inflammatory bowel disease. In some aspects, the therapeutic agent useful for treating inflammatory bowel disease can be selected from one of the following classes of compounds: 5-aminosalicyclic acids, corticosteroids, thiopurines, tumor necrosis factor-α blockers and JAK inhibitors. In some aspects, the treating colorectal cancer can be characterized by the inhibition or reduction of cancer progression.

In some aspects, the m-calpain selective inhibitor can be co-administered together with or separately from a therapeutic agent useful for treating treating colorectal cancer. In some aspects, the therapeutic agent useful for treating colorectal cancer can be selected from one of the following classes of compounds: antineoplastic agents, thymidylate synthase inhibitors, topiosomerase inhibitors, multi-kinase inhibitors, endothelial growth factor receptor (EGFR) inhibitors, vascular endothelial growth factor receptor (VEGF) inhibitors, NFkB inhibitors, angiogenesis inhibitors, anti-metabolites, and anti-cytokine inhibitors.

The growth of tumor cells or a colony of tumor cells can be inhibited by contacting said tumor cells with an effective amount of an selective inhibitor of m-calpain. In some aspects, the tumor cells can comprise colorectal cancer cells.

Inflammation of the colon can be reduced by administering to a subject in need thereof, a therapeutically effective amount of a pharmaceutical composition comprising a m-calpain selective inhibitor, or a pharmaceutically acceptable salt, hydrate, or solute thereof.

The level of m-calpain activity can be reduced in colorectal cancer cells or other cells in the colon by administering to a subject a therapeutically effective amount of a pharmaceutical composition comprising a m-calpain selective inhibitor, or a pharmaceutically acceptable salt, hydrate, or solute thereof. The effective dose of the m-calpain specific inhibitor to do so can be administered at a dose ranging from 0.01 mg/kg to about 1 mg/kg, from about 0.02 mg/kg to about 50 mg/kg, from about 0.1 mg/kg to 50 mg/kg, from about 0.1 mg/kg to 5 mg/kg, from about 0.1 mg/kg to 2 mg/kg, or from about 0.1 mg/kg to 1 mg/kg. The therapeutically effective dose may be, for example, about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, 30, 35, 40, 45 or about 50 mg/kg, or any range in between any two of the recited doses. In some aspects, the dose will be 0.08 mg/kg to about 0.5 mg/kg, from about 0.08 to about 0.24 mg/kg, or from about 0.24 to about 0.85 mg/kg. In one aspect, the effective dose of the m-calpain inhibitor can be given in one or more doses, with the therapeutically effective dose of, for example, 0.08, 0.24, 0.5, 0.75, 1.0, or 1.25 mg/kg for each dose. In one embodiment, the dose is administered by a delivery route selected from the group consisting of intraperitoneal, intradermal, intramuscular, intraperitoneal, intravenous, topical, subcutaneous, anal, or epidural routes. In one embodiment, the one or more effective doses of the m-calpain inhibitor can be administered orally, intravenously, intramuscularly, or subcutaneously. In one embodiment, the one or more effective doses of the m-calpain inhibitor can be administered orally. In one aspect, the one or more effective doses of the m-calpain inhibitor can be administered intravenously. In certain embodiments, the one or more effective doses of the m-calpain inhibitor can be administered subcutaneously. In one embodiment, the one or more effective doses of the m-calpain inhibitor can be administered anally.

In some aspects, the m-calpain selective inhibitor can be administered as a pharmaceutical composition. In some aspects the pharmaceutical composition may be, for example, an immediate release formulation or a controlled release formulation, for example, a delayed release formulation. The delayed release formulation may be used for drug delivery to the colon, which reduces the required therapeutically effective dose compared to systemic dosages. In some aspects administration in a delayed release formulation may reduce side effects and/or improve safety and/or efficacy. In some aspects, the therapeutically effective amount of the m-calpain specific inhibitor administered in a delayed release formulation can be from about 0.1 ng/kg to 4.0 mg/kg, from about 0.5 ng/kg to about 0.5 mg/kg, from about 1.0 ng to about 100 ug/kg, from about 10 ng/kg to about 10 ug/kg, from about 100 ng/kg to about 1 ug/kg, from about 1 mg/kg to about 100 mg/kg, or from about 10 mg/kg to about 1000 mg/kg. In some aspects, the pharmaceutical composition can comprise a formulation further comprising a particle.

This disclosure also describes a kit, comprising a first composition comprising an effective amount of a selective m-calpain inhibitor wherein the selective m-calpain inhibitor is Calpain Inhibitor IV, or a pharmaceutically acceptable salt, hydrate, or solute thereof; and a second composition a therapeutic agent useful for treating inflammatory bowel disease or colorectal cancer, or a pharmaceutically acceptable salt, hydrate, or solute thereof, together with instructions for administering the first composition and the second composition to a patient suffering from colitis. In some aspects, the first and second composition of the kit can be administered in combination, can be administered simultaneously, can be administered separately, can be administered sequentially, or can be administered in a controlled manner.

### Inhibiting the Growth of Tumor Cells

In some aspects, the selective inhibitor of m-calpain can inhibit the growth of tumor cells or a colony of tumor cells comprising a method of contacting said tumor cells with an effective amount of a selective inhibitor of m-calpain. The tumor cells can comprise colorectal cancer cells. In some aspects, the selective m-calpain inhibitor is a modified peptide that comprises at least one partial leucine moiety and an alkyl halide group. In some aspects, the modified peptide is Calpain Inhibitor IV. In some aspects, the m-calpain selective inhibitor is an irreversible inhibitor of m-calpain. In some aspects, the m-calpain selective inhibitor irreversibly inhibits m-calpain with a k₂ rate constant greater than about 28,000 M⁻¹s⁻¹, or greater than 25,000 M⁻¹s⁻¹, or greater than 20,000 M⁻¹s⁻¹, or greater than 15,000 M⁻¹s⁻¹, or greater than 10,000 M⁻¹s⁻¹ or greater than 5,000 M⁻¹s⁻¹. In some aspects, the m-calpain selective inhibitor is a reversible inhibitor of m-calpain. In some aspects, the m-calpain selective inhibitor reversibly inhibits m-calpain with a IC₅₀ of less than about 100 nM, or less than 50 nM, or less than 40 nM, or less than 30 nM, or less than 20 nM, or less than 10 nM, or less than 1 nM. In some aspects, the m-calpain selective inhibitor inhibits or binds to m-calpain more than μ-calpain. In some aspects, the m-calpain selective inhibitor is formulated to preferentially release in the colon.

### Biochemical Mechanisms of Colitis and Colorectal Cancer

In some aspects of this invention, colorectal cancer or inflammatory bowel disease can be treated by administering to a subject in need thereof, a therapeutically effective amount of a pharmaceutical composition comprising a m-calpain selective inhibitor, or a pharmaceutically acceptable salt thereof.

The complexity of IBD and colorectal cancer require multi-targeted intervention strategies based on the underlying pathophysiology. Immunomodulatory therapeutics for IBD and colorectal cancer have mainly targeted secreted proinflammatory mediators or the actions of their receptors like tumor necrosis factor-α (TNF-α) blockers and selective JAK inhibitors, respectively (Peyrin-Biroulet L, Oussalah A, Williet N, et al., Gut; 60: 930-936, 2011; Van Rompaey L, Galien R, van der Aar E M, et al., J Immunol; 191: 3568-3577, 2013). However, one-third of IBD patients do not respond to anti-TNF-α and the treatment and the surgery-sparing effect of these medications are still unclear (Sokol H., Seksik P., Cosnes J., Current opinion in gastroenterology, 2014; Rutgeerts P., Sandborn W. J., Feagan B. G., et al. Infliximab for induction and maintenance therapy for ulcerative colitis. The New England journal of medicine; 353: 2462-2476, 2005). Calpastatin is an endogenous inhibitor of both isoforms of calpain enzymes. Calpastatin increases as a physiologic response to calpain-driven inflammation and mechanistic investigations reveal a role for calpastatin in limiting macrophage activation and inflammatory pathology during colitis. Calpastatin prevents the hyperactivation of macrophages and NFκB driven inflammatory mediator production during colitis (Huang Z., Rose A. H., Hoffmann F. W., et al., supra; Huang Z., Hoffmann F. W., Norton R. L., et al., The Journal of biological chemistry 286: 34830-34838, 2011).

As recognized herein, the central role of calpastatin in limiting inflammation suggests that intervention with a synthetic calpain-2 inhibitor may assist the endogenous response to inflammation and thereby serve as an effective approach to treating IBD and preventing colorectal cancer. As recognized herein, a synthetic inhibitor to calpain enzymes would be a preferable inhibitor to calpain enzymes as calpastatin is involved in the regulation of many cell-cycle events, and so an increase in calpastatin to inhibit calpain may hit off-target pathways leading to an undesirable side-effects in a subject so treated. Some limited data suggest that a synthetic pan-calpain inhibitor may reduce the severity of experimentally induced acute colitis in rats (Cuzzocrea S., McDonald M. C., Mazzon E., et al., Gut; 48: 478-488, 2001). However, this particular reference was deemed non-enabling by others in the art because of flaws in experimental design such as pretreatment of the rats with the inhibitor prior to induction of acute colitis and lack of chronic colitis conditions which limited interpretation of the data (Ballinger A., Azooz O., Gut 50: 440-441, 2002). The inventors have recognized that because of the central role m-calpain plays in promoting macrophage activation and inflammatory pathology, a specific inhibitor to m-calpain will treat both colitis and colorectal cancer (“colitis-associated cancer”, or “CAC”).

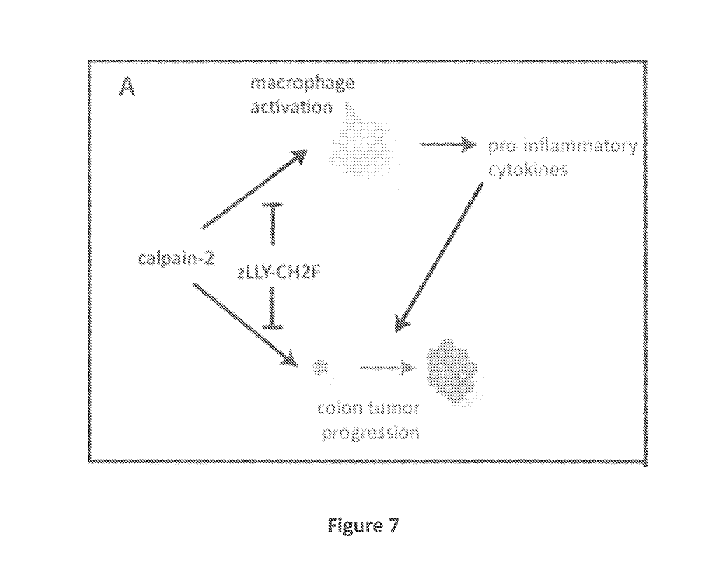

The calpain protease system and its dynamic role in macrophage activation represent a promising target of potential drug intervention for inflammation driven disease. The inventors have recognized that a selective m-calpain inhibitor would suppress the pathology of colitis in the AOM/DSS model. The inventors have made the surprising discovery that a selective m-calpain inhibitor acts by two mechanisms to treat colitis and CAC (FIG. 7). By both reducing inflammation and directly inhibiting CAC cell proliferation, a selective m-calpain inhibitor will have a very strong impact on treating CAC and colitis. Furthermore, initiating intervention after the appearance of symptoms was also surprisingly discovered to still be effective in providing protection against colitis severity and CAC development.

Systematic delivery of the selective m-calpain inhibitor results in major anti-inflammatory effects through inhibiting NFκB signaling in intestinal macrophages. The inhibition of m-calpain results in reduced NFκB translocation and inflammatory cytokine secretion by activated BMDM. Other approaches have focused on inhibiting inflammatory mediators downstream of NFκB translocation. Inflammatory cytokines such as TNFα have been shown to increase in intestinal tissues and peripheral phagocytes of patients with IBD (Zipperlen K., Peddle L., Melay B., Hefferton D., Rahman P., Hum Immunol. 66:56-9, 2005). This has led to the development of monoclonal antibodies against TNF-α for treating inflammation in IBD and other inflammatory disorders (Lapadula G., et al., Int J Immunopathol Pharmacol. 27(1 Suppl):33-48, 2014), although some patients do not respond to this treatment and other inflammatory mediators may contribute to this effect (Rutgeerts P., et al., N Engl J Med. December 8; 353(23):2462-76, 2005; Bank S., et al., Pharmacogenomics J. April 29. doi: 10.1038/tpj.2014.19, 2014). The method of treating colitis or CAC can be extended to not just using a m-calpain specific inhibitor alone, but a coadministration course with another inflammatory mediator or agent used to treat colorectal cancer so as to target a wider population or achieve a more efficacious result during the course of a treatment of a subject.

Calpains catalytically modulate a variety of target proteins including cytoskeletal proteins, membrane receptors, calmodulin binding proteins, G proteins, protein kinase C, other enzymes involved in signal transduction, and transcription factors (Hendry, L. & John, S. Eur. J. Biochem. 271, 4613-4620, 2004). Thus, using a non-specific calpain inhibitor for reducing inflammatory mediators may induce side-effects that depend on other calpain functions. The inventors invented a treatment to use a m-calpain specific inhibitor to reduces side effects by leaving μ-calpain activity intact. The inventors also appreciated that gut-targeted delivery mechanisms further reduce side effects and incorporated this into one formulation of the invention. Enhanced intestinal-specific delivery of the m-calpain inhibitor is an attractive means for concentrating the effects of therapeutics in the intestinal tissues (Youngren S R, Tekade R K, Gustilo B, et al. BioMed research international; 2013: 858946; Youngren S R, Mulik R, Jun B, et al. AAPS PharmSciTech, 14: 1012-1024, 2013; Wang B, Zhuang X, Deng Z B, et al. Molecular therapy: the journal of the American Society of Gene Therapy, 22: 522-534, 2014). The use of compound-encapsulated nanoparticles is one such method by which targeted delivery of the selective m-calpain inhibitor can be achieved so as to reduce the inhibition of non-target functionality.

The anti-inflammatory effects of the m-calpain specific inhibitor are consistent with the anti-inflammatory effects of the endogenous calpain inhibitor, calpastatin, and suggest that m-calpain is the predominant isoform of this enzyme family driving macrophage hyperactivation and severe colitis in this model. However, the inventors made the surprising discovery that the m-calpain specific inhibitor zLLY-CH2F directly affects NFκB nuclear localization in colorectal cancer cell lines and their proliferative capacity. Calpain activity has been linked to chemotherapeutic resistance in colorectal cancer, esophageal cancer, and melanoma (Leloup, L. & Wells, A. Expert Opinion on Therapeutic Targets 15, 309-323, 2011; Ho, W., Pikor, L., Gao, Y., Elliott, B. E. & Greer, P. A. J Biol Chem 287, 15458-15465, 2012; Raimbourg, Q. et al. PLoS ONE 8, e60469, 2013). This indicates that the use of a m-calpain specific inhibitor can treat cancer types other than colorectal cancer. The present invention also relates to the use of a m-calpain specific inhibitor capable of treating esophageal cancer and melanoma or other cancers in which calpain activity mediates chemotherapeutic resistance. In melanoma, the correlation of calpain activity reduction has been extended to survival outcomes with tumors expressing high levels of m-calpain resulting in shorter survival for patients (Raimbourg, supra; M [symbol] ynarczuk-Bia [symbol] y, I. et al., Cancer Res 66, 7598-7605, 2006). Importantly, calpain proteolytic activity has been shown to increase NF-κB signaling in melanoma cancer cells and treatment with a non-specific calpain inhibitor was able to attenuate this signaling in cisplatin resistant cells (M [symbol] ynarczuk-Bia [symbol] y, supra). Other efforts have demonstrated a correlation between calpain expression and drug resistant esophageal cancer (Liu, T.-L. et al. Apoptosis 11, 1025-1037, 2006). These correlations indicate the involvement of the calpain/calpastatin system in carcinogenesis and tumor progression. The inhibition of m-calpain activity by m-calpain selective inhibitors protects from chronic inflammation in IBD and can prevent pathology of the gut arising from this chronic inflammation as well as directly inhibit growth of the colon cancer cells themselves. The combination of effects on the inflammation and the colorectal cancer indicate this approach is an effective inflammation treatment.

Structure and Properties of Selective m-Calpain Inhibitor Compounds

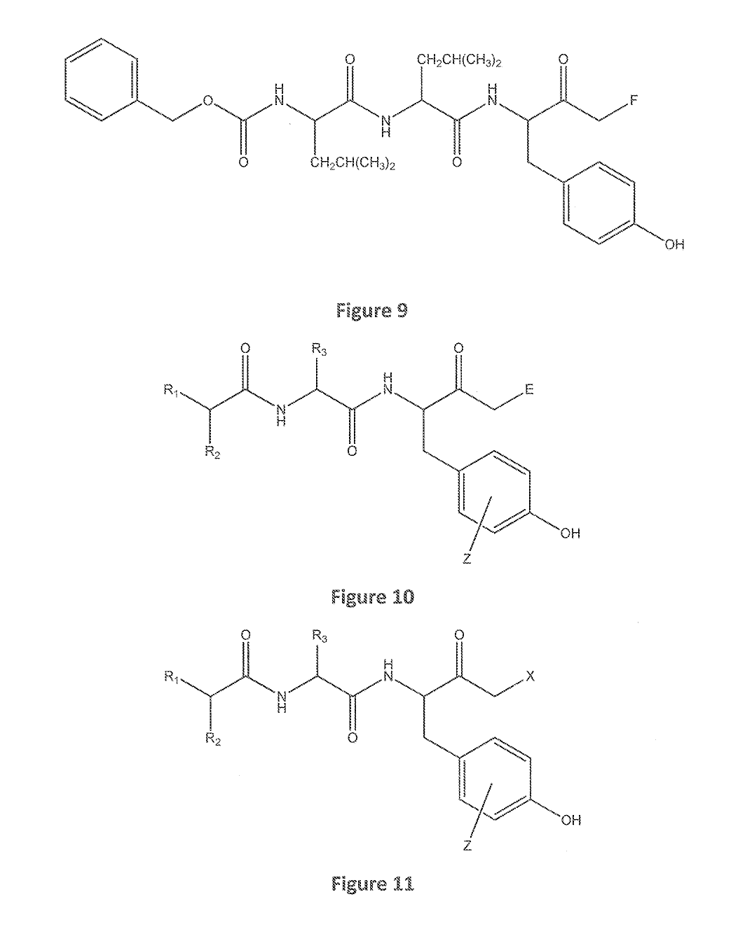

Calpain inhibitors include: Calpain Inhibitor I, Calpain Inhibitor II, Calpain Inhibitor III, Calpain Inhibitor, IV, Calpain Inhibitor V, Calpain Inhibitor, VI, Calpain Inhibitor VII, Calpain Inhibitor, VIII, Calpain Inhibitor IX, Calpain Inhibitor X, Calpain Inhibitor X, and Calpain Inhibitor XII. A variety of other synthetic calpain inhibitors with selectivity (“preferentiality”) to m-calpain or μ-calpain are described in Markus Pietsch M., Chua K. C. H., Andrew A., Current Topics in Medicinal Chemistry, 10, 270-293, 2010. Many of these compounds are reversible inhibitors of m-calpain because no permanent bond is formed from the free cysteine of calpain in the inhibitor binding pocket (Moldoveanu, T.; Campbell, R. L.; Cuerrier, D.; Davies, P. L., J. Mol. Biol., 343, 1313-1326, 2004; Cuerrier, D.; Moldoveanu, T.; et al. J. Biol. Chem., 282, 9600-9611, 2007). Z-LLY-CH2F (“Calpain Inhibitor IV”), as shown in FIG. 9 is a synthetic compound which is both selective for m-calpain and an irreversible inhibitor of m-calpain. In some aspects, the selective m-calpain inhibitor of the invention can have the central “core” of Z-LLY-CH2F, but with varied substituent groups R₁, R₂, R₃, Z, and X, as shown in FIG. 10. R₁, R₂, R₃, and Z can be alkyl, branched alkyl (“alkyl”), aryl, heteroaryl, hydrogen, halo, ester, ether, alkoxy ether, alkoxy ester, alkoxy reverse ester, thiol ether, or alkoxy thiol ether. The “E” in FIG. 10 can be an electrophilic group, as a non-limiting example: aldehyde, hemiacetal (in prodrug form), epoxy, or a leaving group. FIG. 11 depicts the molecular structure of a preferred class of embodiments wherein X is a halogen or conjugate base of a strong acid, and thus a leaving group (CI, Br, F, I, At, sulfate, methyl sulfate, phosphate, alkyl sulfate, aryl sulfate, substituted aryl sulfate). The structure can have at least one partial leucine moiety and an alkyl halide group.

In some aspects, the m-calpain selective inhibitor (or “selective m-calpain inhibitor,” or “selective calpain-2 inhibitor”) can be a modified peptide that comprises at least one partial leucine moiety and an electrophilic moiety. In some aspects, the electrophilic moiety is an alkyl halide group. In some aspects, the m-calpain selective inhibitor is ZZ-LLY-CH2F (“Calpain Inhibitor IV”, or ZZ-LLY-FMK), the structure of which is shown in FIG. 9.

In some aspects, the m-calpain selective inhibitor can be an irreversible inhibitor of m-calpain. The inhibition is made reversible by the nucleophilic attack of the active cysteine in m-calpain to the electrophilic moeity of the m-calpain selective inhibitor to form a covalent bond (Moldoveanu, T.; Campbell, R. L.; Cuerrier, D.; Davies, P. L. J. Mol. Biol., 343, 1313-1326, 2004; Carragher, N. O., Curr. Pharm. Des., 12, 615-638, 2006). In some aspects, the m-calpain selective inhibitor can irreversibly inhibit m-calpain with a k2 rate constant of about 28,900 M-1 s-1, or greater than 25,000 M-1 s-1, or greater than 20,000 M-1s-1, or greater than 15,000 M-1s-1, or greater than 10,000 M-1s-1 or greater than 5,000 M-1s-1. As the compound ZZ-LLY-CH2F is not known to inhibit μ-calpain, its off-target inhibition rate for this protein cannot be ascertained. However, ZZ-LLY-CH2F can inhibit the off-target protein cathespin L with a rate constant k2 of 680,000 M⁻¹s⁻¹ (Li, Q.; Hanzlik, R. P.; Weaver, R. F.; Schoenbrunn, E., Biochemistry, 45:701-708, 2006). Thus, the ratio of inhibition rates k2 of the on-target m-calpain to an off-target protein can be around 28,900/680,000, or about 0.041. The ratio of inhibition rates k2 of the on-target m-calpain to an off-target protein can be greater than about 0.5, or greater than 0.1, or greater than 0.05, or greater than 0.01.

In some aspects, the m-calpain selective inhibitor can be a reversible inhibitor of m-calpain. In some aspects, the m-calpain selective inhibitor selectivity can be defined as inhibiting or binding to m-calpain significantly more than to μ-calpain. The ratio of the IC₅₀ of the m-calpain selective inhibitor to the off-target μ-calpain compared to the IC₅₀ of the m-calpain selective inhibitor to the on-target m-calpain can be greater than 10, greater than 20, or greater than 100.

In some aspects, the m-calpain selective inhibitor can reversibly inhibit m-calpain with a IC₅₀ of less than about 100 nM, or less than 50 nM, or less than 40 nM, or less than 30 nM, or less than 20 nM, or less than 10 nM, or less than 1 nM.

### Pharmaceutical Formulation/Compositions and Uses

The m-calpain inhibitors for use in the therapeutic treatment (including prophylactic treatment) of mammals including humans, may be formulated as a pharmaceutical composition. In one embodiment, this invention provides pharmaceutical compositions comprising a compound of this invention in association with a pharmaceutically acceptable diluent or carrier.

Formulations of this invention may be prepared by mixing a m-calpain inhibitor compound and a carrier, diluent or excipient. Suitable carriers, diluents and excipients include, for example, materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include sterile water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the m-calpain inhibitor compound (e.g., complex with a cyclodextrin derivative or other complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The formulation may be prepared by applying a coating to a central core containing the pharmaceutical composition. The coating can be an enteric coating so as to prevent the premature release of the m-calpain selective compound. The coating may further comprise the m-calpain selective compound. The coating may be applied to the core by spray-drying, lyophilization (freeze-drying), dip-coat-dry, sputtering, hot melt extrusion, chemical vapor deposition, or coagulation. The core can be a pellet, particle, bead, gel, or film. The core can be made by milling, micronizing, lyophilization (free-drying), extrusion, or compression of the m-calpain selective compound with appropriate binders.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers include, for example, materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a m-calpain inhibitor compound having the desired degree of purity may optionally be mixed with pharmaceutically acceptable diluents, carriers, excipients or stabilizers (Remington's Pharmaceutical Sciences (1980) 16th edition, Osol, A. Ed., and herein incorporated by reference), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

Controlled-release preparations of m-calpain inhibitor compositions are also provided, and may be prepared as described herein. As an example, one or a plurality of coatings containing the m-calpain inhibitor composition can be applied to a core which contains the pharmaceutical composition that delays the release of the drug from the core after administration for a certain period of time. Suitable examples of delayed-release preparations include semipermeable matrices of solid hydrophobic polymers containing the m-calpain inhibitor composition, wherein said matrices are in the form of shaped articles, e.g., films, pills, lozenges, spheres, or microcapsules. Examples of sustained-release matrices include polyesters, hydrogels (e.g., poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid. Other materials that can comprise the one or a plurality of coatings for are known under the trade name EUDRAGIT® L12.5, L100, or EUDRAGIT® S12.5, S100, and several commercially available enteric dispersion systems (e.g., EUDRAGIT® L30D55, EUDRAGIT® FS30D, EUDRAGIT® L100-55, EUDRAGIT® S100 (Rohm Pharma), KOLLICOAT® MAE30D and 30DP (BASF), ESTACRYL® 30D (Eastman Chemical), AQUATERIC® and AQUACOAT® CPD30 (FMC)). The foregoing is a list of possible materials, but one of skill in the art would appreciate that there are other such materials that would meet the objectives of the present invention of providing for a delayed release profile including tailoring release based on the ambient pH environment, temporal considerations and other factors. The in vivo delay in the release can be tailored to a particular application, including the lower intestine, preferably in the colon.

The compound of this invention for use herein is preferably sterile. In particular, formulations to be used for in vivo administration must be sterile. Such sterilization is readily accomplished by filtration through sterile filtration membranes.

The compound ordinarily can be stored as a solid composition, a lyophilized formulation or as an aqueous solution (e.g. in saline).

Formulations of a m-calpain inhibitor compound suitable for oral administration may be prepared as discrete units such as pills, capsules, cachets or tablets each containing a predetermined amount of a compound of the m-calpain inhibitor compound.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of the selective m-calpain inhibitor compound intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, e.g., inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Aqueous suspensions of m-calpain inhibitor compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of m-calpain inhibitors may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 0.0001 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 0.01 to 10,000 μg of the active ingredient per milliliter of solution in order that infusion of a suitable volume at a rate of about 30 mL/hr can occur.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising, e.g. cocoa butter or a salicylate.

The formulations may be packaged in unit-dose or multi-dose containers, e.g. sealed ampoules and vials, and may be stored in a freeze-dried (lyophilized) condition requiring only the addition of the sterile liquid carrier, e.g., water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions of the invention comprising a m-calpain inhibitor compound will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors considered to medical practitioners. In addition to the compounds and salt forms provided herein, the invention includes pharmaceutical compositions, including tablets, capsules, solutions, and suspensions for parenteral and oral delivery forms and formulations, comprising a pharmaceutically acceptable carrier and therapeutically effective amounts of one or more of the m-calpain inhibitors herein provided. Inhibitors of m-calpain pharmaceutical compositions can include salts and hydrates.

In human and animal therapy for the treatment of cancer, for example in the treatment of cancer and other related disorders, diseases and conditions noted herein, the compounds and their crystal forms described and provided herein, their pharmaceutically acceptable salts, and pharmaceutically acceptable solvates of either entity, can be administered alone, but will generally be administered in admixture with a pharmaceutical carrier selected with regard to the intended route of administration and standard pharmaceutical practice. Preferably, they are administered orally in the form of tablets containing pharmaceutically acceptable excipients, such as starch or lactose, or in capsules or ovules either alone or in admixture with excipients, or in the form of elixirs, solutions or suspensions containing flavoring or coloring agents. They can also be injected parenterally, for example, intravenously, intramuscularly or subcutaneously. For parenteral administration, they are best used in the form of a sterile aqueous solution which may contain other substances, for example enough salts or monosaccharides to make the solution isotonic with blood. For buccal or sublingual administration they may be administered in the form of tablets or lozenges which can be formulated in a conventional manner.

The administration of the m-calpain inhibitor composition can comprise further coadministration of the m-calpain selective inhibitor, or a pharmaceutically acceptable salt thereof, and a therapeutically effective amount of an agent useful for treating inflammatory bowel disease and/or inflammation of the colon can be administered sequentially or simultaneously. The m-calpain selective inhibitor, or a pharmaceutically acceptable salt thereof, and the therapeutically effective amount of an agent useful for treating inflammatory bowel disease can be administered within about one hour of each other, within about one day or each other, or within about one week of each other, or within about one month of each other. In some aspects of the coadministration, the m-calpain selective inhibitor can be administered first.

In some aspects, the therapeutic agent useful for treating inflammatory bowel disease can be selected from one of the following classes of compounds: 5-aminosalicyclic acids, corticosteroids, thiopurines, tumor necrosis factor-α blockers and JAK inhibitors. In some aspects, the therapeutic agent useful for treating inflammatory bowel disease can be selected from one or more of the following agents: Prednisone, Humira, Lialda, Imuran, Sulfasalazine, Pentasa, Mercaptopurine, Azathioprine, Apriso, Simponi, Enbrel, Humira Crohn's Disease Starter Pack, Colazal, Budesonide, Azulfidine, Purinethol, Proctosol HC, Sulfazine EC, Delzicol, Balsalazide, Hydrocortisone acetate, Infliximab, Mesalamine, Proctozone-HC, Sulfazine, Orapred ODT, Mesalamine, Azasan, Asacol HD, Dipentum, Prednisone Intensol, Anusol-HC, Rowasa, Azulfidine EN-tabs, Veripred 20, Uceris, Adalimumab, Hydrocortisone, Colocort, Pediapred, Millipred, Azathioprine injection, Prednisolone sodium phosphate, Flo-Pred, Aminosalicylic acid, ProctoCream-HC, 5-aminosalicylic acid, Millipred DP, Golimumab, Prednisolone acetate, Rayos, Proctocort, Paser, Olsalazine, Procto-Pak, Purixan, Cortenema, Giazo, Vedolizumab, Entyvio, Micheliolide, and Parthenolide.

In some aspects, treating colorectal cancer can further comprise the treatment with a m-calpain selective inhibitor and a therapeutically effective amount of an agent useful for treating colorectal cancer. Administering the two compositions can be simultaneous or sequential. The sequential coadministration of the two compositions can be within about one hour of each other, within about one day or each other, or within about one week of each other, or within about one month of each other. In some aspects, the m-calpain selective inhibitor can be administered first, before the administration of other agents.

In some aspects, the therapeutic agent useful for treating colorectal cancer can be selected from one of the following classes of compounds: antineoplastic agents, thymidylate synthase inhibitors, topiosomerase inhibitors, multi-kinase inhibitors, endothelial growth factor receptor (EGFR) inhibitors, vascular endothelial growth factor receptor (VEGF) inhibitors, NFkB inhibitors, angiogenesis inhibitors, anti-metabolites, and anti-cytokine inhibitors. In some aspects, the therapeutic agent useful for treating colorectal cancer can be selected from one of the following compositions useful for treating colorectal cancer: Adrucil (Fluorouracil), Aclarubicin, Avastin (Bevacizumab), Betaseron (interferon beta-1b), BIBF-1120 (3-Z-[I-(4-(N-((4-methyl-piperazin-1-yl)-methylcarbonyl)-N-methyl-amino)-anilino)-I-phenyl-methylene]-6-methoxycarbonyl-2-indolinone), BIBW 2992 (3-Z-[I-(4-(N-((4-methyl-piperazin-1-yl)-methylcarbonyl)-N-methyl-amino)-anilino)-I-phenyl-methylene]-6-methoxycarbonyl-2-indolinone), Adriamycin, Daunomycin, Aclarubicin, Amrubicin, Idarubicin, Epirubicin, Pirarubicin, Dacarbazine, Mitoxantrone Bevacizumab, Camptosar (Irinotecan Hydrochloride), Capecitabine (Xeloda), Cisplatin, Carboplatin, Satraplatin, analogues of Cisplatin, Efudex (Fluorouracil), Eloxatin (Oxaliplatin), Erbitux (Cetuximab), Fluorouracil, Irinotecan Hydrochloride, Leucovorin Calcium, Oxaliplatin, Panitumumab, Regorafenib, Stivarga (Regorafenib), Vectibix (Panitumumab), Wellcovorin (Leucovorin Calcium), Zaltrap (Ziv-Aflibercept), CAPDX, FOLFIRI FOLFIRI-BEVACIZUMAB, FOLFIRI-CETUXIMAB, FOLFOX, FU-LV, and XELOX.

The therapeutically effective dose of the m-calpain specific inhibitor can be administered at a dose ranging from 0.01 mg/kg to about 1 mg/kg, from about 0.02 mg/kg to about 50 mg/kg, from about 0.1 mg/kg to 50 mg/kg, from about 0.1 mg/kg to 5 mg/kg, from about 0.1 mg/kg to 2 mg/kg, or from about 0.1 mg/kg to 1 mg/kg. The therapeutically effective dose may be, for example, about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, 30, 35, 40, 45 or about 50 mg/kg, or any range in between any two of the recited doses. In some aspects, the dose will be 0.08 mg/kg to about 0.5 mg/kg, from about 0.08 to about 0.24 mg/kg, or from about 0.24 to about 0.85 mg/kg. In another aspect, the therapeutically effective dose of the m-calpain inhibitor is given in one or more doses. For example, a therapeutically amount of, for example, 0.08, 0.24, 0.5, 0.75, 1.0, or 1.25 mg/kg for each dose. In one embodiment, the dose is administered by a delivery route selected from the group consisting of intraperitoneal, intradermal, intramuscular, intraperitoneal, intravenous, topical, subcutaneous, anal, or epidural routes. In one aspect, the one or more effective doses of the m-calpain inhibitor are administered orally, intravenously, intramuscularly, or subcutaneously. In one embodiment, the one or more effective doses of the m-calpain inhibitor are administered orally. In one aspect, the one or more effective doses of the m-calpain inhibitor are administered intravenously. In certain embodiments, the one or more effective doses of the m-calpain inhibitor are administered subcutaneously. In one embodiment, the one or more effective doses of the m-calpain inhibitor are administered anally.

In one aspect, the therapeutically effective amount (“dose”) of the m-calpain inhibitor can be administered at a dose ranging from 0.01 microgram/kg to 4 mg/kg. The therapeutically effective dose may be, for example, about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.9, 3.0, 3.1, 3.2, 3.3, 3.4, 3.5, 3.6, 3.7, 3.8, 3.9, 4.0, 5.0, 6.0, 7.0, 8.0, 9.0, or about 10.0 microgram/kg, or any range in between any two of the recited doses. In some aspects, the dose will be 0.08 microgram/kg to about 0.5 microgram/kg, from about 0.08 to about 0.24 microgram/kg, or from about 0.24 to about 0.5 microgram/kg. In another aspect, the effective dose of the m-calpain inhibitor is given in one or more doses.

In some aspects, the therapeutically effective amount comprises one or more effective doses of the pharmaceutical composition comprising the m-calpain selective inhibitor. In some aspects, the m-calpain selective inhibitor can be administered as a pharmaceutical composition. In some aspects the pharmaceutical composition can comprise a formulation further comprising one or a plurality of particles. In some aspects the pharmaceutical composition may be, for example, an immediate release formulation or a controlled release formulation, for example, a delayed release particle.

In some aspects, the particle can be a particle selected from one of the following: polylactide (PLA) nanoparticles, poly-DL-lactic acid (PDLLA) microspheres, poly (lactic acid) nanoparticles, chitosan-modified poly (D,L-lactide-co-glycolide) nanospheres (CS-PLGA NSs), chitosan-alginate coated nanoparticle, solid lipid nanoparticles (SLNs), grapefruit-derived nanoparticles (GDNs), silicon nanoparticles, polylactic-co-glycolic acid (PLGA) nanoparticles, pH-sensitive Eudragit P-4135F nanoparticles, thioketal nanoparticles (TKNs) made from the polymer poly-PPADT (1,4-phenyleneacetone dimethylene thioketal), lipopolysaccharides (LPS), and type B gelatin enclosed in poly(e-caprolactone) (PCL) microspheres. In some embodiments, the particle is a chitosan-alginate coated nanoparticle. Selective m-calpain inhibitors also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are disclosed in Remington's Pharmaceutical Sciences, supra).

In some aspects, the therapeutically effective amount of the compound when administered in a particle formulation can be from about 0.1 ng/kg to 4.0 mg/kg, from about 0.5 ng/kg to about 0.5 mg/kg, from about 1.0 ng to about 100 ug/kg, from about 10 ng/kg to about 10 ug/kg, from about 100 ng/kg to about 1 ug/kg, from about 1 mg/kg to about 100 mg/kg, or from about 10 mg/kg to about 1000 mg/kg.

In some aspects, the m-calpain selective inhibitor is formulated to preferentially release in the colon.

### Articles of Manufacture/Kits

In another embodiment of the invention, an article of manufacture, or “kit”, containing materials useful for treating the diseases and disorders described above is provided. The kit comprises a container comprising a selective m-calpain inhibitor. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container may hold a selective m-calpain inhibitor or a formulation thereof which is effective for treating the condition and may have a sterile access port (e.g., the container may be an intravenous solution bag or a vial having a stopper pierceable by a hypodermic injection needle). At least one active agent in the composition is a selective m-calpain inhibitor. The label or package insert indicates that the composition is used for treating the condition of choice, such as cancer. In addition, the label or package insert may indicate that the patient to be treated is one having a disorder such as a hyperproliferative disorder. In one embodiment, the label or package inserts indicates that the composition comprising a selective m-calpain inhibitor can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the selective m-calpain inhibitor and, if present, the second pharmaceutical formulation. For example, if the kit comprises a first composition comprising a selective m-calpain inhibitor, and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a selective m-calpain inhibitor composition, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, e.g. in the form of numbers, letters, or other markings or with a calendar insert, designating the days in the treatment schedule in which the dosages can be administered.

In certain other embodiments wherein the kit may comprise a selective m-calpain inhibitor and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

The invention includes an article of manufacture comprising packaging material containing one or more dosage forms containing selective m-calpain inhibitors provided herein, wherein the packaging material has a label that indicates that the dosage form can be used for a subject having or suspected of having or predisposed to any of the diseases, disorders and/or conditions described or referenced herein. Such dosage forms include, for example, tablets, capsules, solutions and suspensions for parenteral and oral delivery forms and formulations.

In one aspect, the kit can comprise a first composition comprising an effective amount of a selective m-calpain inhibitor wherein the selective m-calpain inhibitor is Calpain Inhibitor IV, or a pharmaceutically acceptable salt thereof; and a second composition a therapeutic agent useful for treating inflammatory bowel disease or colorectal cancer, or a pharmaceutically acceptable salt thereof, together with instructions for administering the first composition and the second composition to a patient suffering from colitis is described herein. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some aspects, the first and second (and optionally, third) compositions of the kit can be administered in combination, can be administered simultaneously, can be administered separately, can be administered sequentially, or can be administered in a sustained manner.

Methods of Treatment with m-Calpain Selective Compounds

Selective inhibitors of m-calpain of the present invention are useful for treating hyperproliferative diseases, conditions and/or disorders including, but not limited to, cancer and colitis. Accordingly, an aspect of this invention includes methods of treating, or preventing, diseases or conditions that can be treated or prevented by inhibiting m-calpain. In one embodiment, the method comprises administering to a subject, in need thereof, a therapeutically effective amount of a compound of a selective m-calpain inhibitor, or pharmaceutically acceptable salt thereof. In one embodiment, a human patient is treated with a selective m-calpain inhibitor and a pharmaceutically acceptable carrier, adjuvant, or vehicle, wherein said selective m-calpain inhibitor is present in an amount to treat cancer and/or detectably inhibit m-calpain activity.

Cancers which can be treated according to the methods of this invention include, but are not limited to, colorectal, rectal, glioma, glioblastoma, neuroblastoma, breast, ovary, cervix, prostate, testis, genitourinary tract, esophagus, larynx, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, buccal cavity and pharynx (oral), lip, tongue, mouth, pharynx, small intestine, and large intestine.

Selective inhibitors of m-calpain of the present invention are useful for inhibiting the growth of tumor cells or a colony of tumor cells by contacting said tumor cells with an effective amount of an selective inhibitor of m-calpain. In some aspects, the tumor cells can comprise colorectal cancer cells.

Selective inhibitors of m-calpain of the present invention are useful for reducing inflammation in the colon. The level of m-calpain activity in macrophages is reduced in colorectal cancer cells or other cells in the colon which lead to inflammation in the colon.

Selective inhibitors of m-calpain of the present invention are useful for treating inflammatory bowel disease. The inflammatory bowel disease can be one of the following classifications of disease: ulcerative colitis, Crohn's disease, collagenous colitis, lymphocytic colitis, ischaemic colitis, diversion colitis, Behcet's syndrome, infective colitis or indeterminate colitis.

Selective inhibitors of m-calpain of the present invention are useful for treating other cancer types which have increased m-calpain activity, including esophageal cancer and melanoma (Raimbourg, Q. et al. PLoS ONE 8, e60469, 2013; Liu, T.-L. et al. Apoptosis 11, 1025-1037, 2006; Leloup, L. & Wells, A. Expert Opinion on Therapeutic Targets 15, 309-323, 2011). These cancers often exhibit chemotherapeutic resistance due to the increased m-calpain activity. Treatment of the chemotherapeutic resistant cancers can be achieved with the selective m-calpain inhibitor.

Metabolites of Selective m-Calpain Inhibitor Compounds

Also falling within the scope of this invention are the in vivo metabolic products of the synthetic selective m-calpain inhibitors, described herein. Such products may result, e.g., from the condensation, oxidation, reduction, hydrolysis, amidation, deamidation, esterification, deesterification, enzymatic cleavage, and the like, of the administered compound. Accordingly, the invention includes metabolites of synthetic selective m-calpain inhibitors, including compounds produced by a process comprising contacting a compound of this invention with a mammal for a period of time sufficient to yield a metabolic product thereof.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, may be useful in diagnostic assays for therapeutic dosing of the compounds of the invention.

### DEFINITIONS

The practice of some embodiments presented herein will employ, unless otherwise indicated, conventional techniques of chemistry, molecular biology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984; Methods of Enzymology: DNA Structure Part A: Synthesis and Physical Analysis of DNA Methods in Enzymology, Academic Press; and E. M. Shevach and W. Strober, 1992 and periodic supplements, Current Protocols in Immunology, John Wiley & Sons, New York, N.Y. Each of these general texts is herein incorporated by reference.

For the purpose of the current disclosure, the following definitions shall, in their entireties, be used to define technical terms, and to define the scope of the composition of matter for which protection is sought in the claims.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which comprise oxygen, nitrogen, sulfur, or phosphorous, atoms replacing one or more carbons of the hydrocarbon backbone.

The term “aryl” includes groups with aromaticity, including 5- and 6-membered single-ring aromatic groups that may include from zero to four heteroatoms, as well as multicyclic systems with at least one aromatic ring, and also stilbenes (substituted and non-substituted) and vinyl stilbenes (substituted and non-substituted). Examples of aryl groups include benzene, phenyl, pyrrole, furan, thiophene, thiazole, isothiazole, imidazole, triazole, substituted triazoles, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused, or bridged, with alicyclic or heterocyclic rings which are not aromatic, so as to form a multicyclic system (e.g., tetralin, methylenedioxyphenyl).

As used herein, a “subject” refers to an animal that is the object of treatment, observation or experiment. “Animal” includes cold- and warm-blooded vertebrates and invertebrates, such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, apes, and prenatal, pediatric, and adult humans.

As used herein, “preventing” or “protecting” means preventing in whole or in part, or ameliorating, or controlling.

As used herein, the term “treating” refers its meaning as known in the art, and to both therapeutic treatment and prophylactic, or preventative, measures, or administering an agent suspected of having therapeutic potential. The term includes preventative (e.g., prophylactic) and palliative treatment. As used herein, the term “treatment” also includes symptomatic therapy to lessen, alleviate, or mask the symptoms of the disease or disorder, as well as therapy for preventing, lowering, stopping, or reversing the progression of severity of the condition or symptoms being treated. As such, the term “treatment” includes both medical therapeutic treatment of an established condition or symptoms and/or prophylactic administration, as appropriate.

The term “a pharmaceutically effective amount”, as used herein, means an amount of active compound, or pharmaceutical agent, that elicits the biological, or medicinal, response in a tissue, system, animal, or human that is being sought, which includes alleviation or palliation of the symptoms of the disease being treated and/or an amount sufficient to have utility and provide desired therapeutic endpoint. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; cause loss of viability, inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, e.g., by assessing the time to disease progression and/or determining the response rate.

“Solid tumors” generally refers to the presence of cancer of body tissues other than blood, bone marrow, or the lymphatic system.

An effective amount of m-calpain inhibitors to be employed therapeutically will depend, for example, upon the therapeutic objectives, the route of administration, and the condition of the patient. Accordingly, it will be necessary for the therapist to titer the dosage and modify the route of administration, as required to obtain the optimal therapeutic effect. Typically, the clinician will administer m-calpain inhibitors until a dosage is reached that achieves the desired effect. In certain embodiments, the appropriate dosing can be determined based on an amount of m-calpain inhibitors administered per surface area of the affected region.

The term “pharmaceutically acceptable”, as used herein, means that the substance or composition must be compatible chemically and/or toxicologically, with the other ingredients comprising a formulation, and/or the mammal being treated therewith.

The term “cancer” refers to, or describes, the physiological condition in mammals that is typically characterized by unregulated cell growth and/or hyperproliferative activities. A “tumor” comprises one or more cancerous cells. Examples of cancer include, but are not limited to, carcinoma, lymphoma, blastoma, sarcoma. In one embodiment, the cancer is a solid tumor. More particular examples of such cancers include cervical cancer, ovarian cancer, bladder cancer, endometrial or uterine carcinoma, prostate cancer, hepatocellular cancer, gastric or stomach cancer including gastrointestinal cancer, pancreatic cancer, glioblastoma, liver cancer, hepatoma, colon cancer, rectal cancer, colorectal cancer, salivary gland carcinoma, kidney or renal cancer, vulval cancer, hepatic carcinoma, anal carcinoma, and penile carcinoma. In one embodiment, the treatment comprises treatment of solid tumors. In one embodiment, the tumors comprises sarcomas, carcinomas or lymphomas.

A “metabolite” is a product produced through metabolism in the body of a specified compound, or salt thereof. Metabolites of a compound may be identified using routine techniques known in the art, and their activities determined, using tests such as those described herein. Such products may result e.g., from the oxidation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic, or inorganic, salts of a compound of the invention. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis(2-hydroxy-3-naphthoate)) salts. A pharmaceutically acceptable salt may involve the inclusion of another molecule, such as an acetate ion, a succinate ion, or other counter ion. The counter ion may be any organic, or inorganic, moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, e.g., treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, e.g., treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A “solvate” refers to an association, or complex, of one or more solvent molecules and a compound of the invention. Examples of solvents that form solvates include, but are not limited to, water, isopropanol, ethanol, methanol, DMSO, ethylacetate, acetic acid, and ethanolamine.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

### Example 1

### Treating Colitis with Calpain-2 Inhibitor

### Materials and Methods

### AOM/DSS Model.

A mice colony generated from C57BL/6J mice (Jackson Laboratories) was maintained in the University of Hawaii vivarium. The mouse AOM/DSS induced colitis/CAC model was adapted from a previously described protocol (Fenouille, N. et al. J. Pathol. 227, 118-129, 2012). In brief, 8-10 wk old male mice were injected with AOM (A5486, Sigma) (12 mg/kg) on day 0 and were then treated with three 5 day cycles of 2% DSS (CAS#9011-18-1; MP Biomedicals, Santa Ana, Calif.) in drinking water alternated with regular water over 63 days. Mice in the intervention group were interperitonal injected 5 days per week with 0.75 mg/kg calpain-2 inhibitor (Calpain Inhibitor IV, zLLY-CH2F; Millipore EMD part number 208724) beginning on day 8 and then throughout the entire protocol. Control mice were injected with AOM on day 0 and received regular drinking water throughout the protocol. All animal protocols were approved by the University of Hawaii Institutional Animal Care and Use Committee.

### Western Blots, Proliferation Assays, Calpain Activity Assays, and Cytokine Analyses.

Bone marrow derived macrophages (BMDM) were prepared as previously described by methods known to those skilled in the art. On day 5 of culture, 2×10⁶ BMDM were seeded in 6-well plates and the following day were primed for 18 h with TNFα (20 ng/mL), followed by 1 h stimulation with E. faecalis (1 μg/mL; ATCC). Stimulation with E. faecalis was conducted in the presence of the zLLY-CH2F calpain-2 inhibitor (20 μg/mL) or DMSO as a control. Media was removed from treated BMDM, centrifuged at 300×g for 5 min and supernatent analyzed using the Cytometric Bead Array Mouse Inflammation Kit (BD Biosciences, San Jose, Calif.). Cytokine data were analyzed using Flowjo software (Ashland, Oreg.) and GraphPad Prism version 4.0 (GraphPad, La Jolla, Calif.). Cell pellets were lysed and analyzed for IκB and NFκB by Western blot as previously described (Miyazaki, T. et al. Journal of Atherosclerosis and Thrombosis 20, 228-237, 2013). In other experiments, mouse CT-26.WT and human HT-29 colon cancer cells were plated in 96-well plates at 10³ cells/well with 20 μg/mL inhibitor or DMSO, followed by analyses for IκB and NFκB by Western blot as described above for BMDM. For proliferation assays, fresh media with 20 μg/mL inhibitor or DMSO were added every 2 d. Each day 4 wells were quantified using Celltiter MTS reagent (Promega) and the resulting signal was used to generate a proliferation curve. Calpain activity was measured using a Calpain activity assay kit (BioVision, Inc., Milpitas, Calif.) as previously described. Colons were harvested on day 4 and calpain activity measured. Enzyme-linked immunosorbant assay (ELISA) analyses of cecum hemoglobin has been previously described (Miyazaki, T. et al. Journal of Atherosclerosis and Thrombosis 20, 228-237, 2013).

### Imaging of Colon Tissues and Histology

On day 63, colons were dissected, cut longitudinally, and then were spread out on clear plastic. The colons were washed with PBS using transfer pipettes and images captured using an Infinity 2 microscope mounted camera (Lumenara, Madrid, Spain) in line with a Stemi 2000-C dissecting scope (Zeiss, Jena, Germany). Separate images were assembled into continuous colon images using Photoshop software (Adobe Systems Inc.). For histology, colon tissues were washed with PBS and fixed in 10% buffered formalin. Standard H&E staining of paraffin-embedded tissue samples was conducted as previously described (Leloup, L. & Wells, A., Expert Opinion on Therapeutic Targets 15, 309-323, 2011). Five H&E stained sections from each mouse were blindly scored for colitis pathology as previously described (Liu, T., Mendes, D. E. & Berkman, C. E. Int. J. Oncol. 44, 467-472, 2014). Colon cancer pathology was assessed on a four point system as follows: 1=no tumor or dysplasia present; 2=basally oriented nuclei, mild nuclear enlargement, nuclear crowding and hyperchromasia, decreased or loss of intracellular mucin; 3=prominent nuclear stratification, more severe hyperchromasia and pleomorphism, marked architectural distortion; 4=back to back glands with no intervening stroma, dysplastic epithelial cells and invasion of the colonic basement membrane. Images captured using a Zeiss Axioskop 2 Plus upright light microscope and camera.

### Statistical Analyses

Comparison of two means was carried out using an unpaired Student's t test using GraphPad Prism version 4.0 (GraphPad, La Jolla, Calif.). Weight curves and experiments with 3 or more groups were compared by repeated measures one-way ANOVA followed by Tukey post-test. Standard curves and regression analyses were also conducted using GraphPad Prism version 4.0. All comparisons were considered significant at P<0.05.

### Results

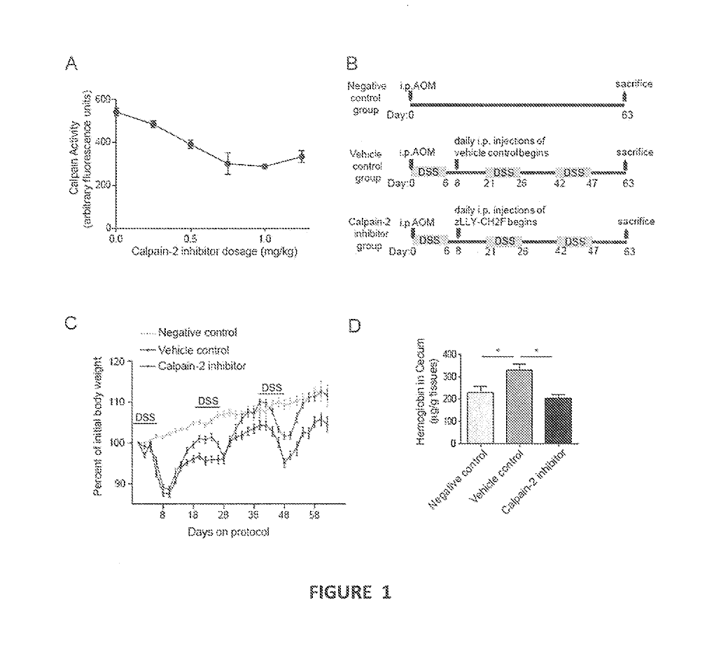

In order to determine an effective in vivo dosage of calpain-2 inhibitor mice were subjected to 2% DSS-treated drinking water combined with interperitonal injections with increasing concentrations of the calpain-2 inhibitor (zLLY-CH2F at 0-1.25 mg/kg), and then calpain activity was measured in the colon tissue. The protocol was carried out for 4 days. Results indicated that at 0.75 mg/kg of calpain-2 inhibitor the maximum amount of calpain activity inhibition was achieved (FIG. 1, panel A). Total calpain activity was measured and reduced by 50%.

An established AOM/DSS model was carried out to determine the effects of intervention with 0.75 mg/kg calpain-2 inhibitor on colitis and CAC (FIG. 1, panel B). Body weights were measured every other day and intervention with the calpain-2 inhibitor was initiated on day 8 after the initial appearance of weight loss. Results showed that the mice treated with the calpain-2 inhibitor had a significant increase in weight recovery after each administration of DSS compared to the weights of the mice injected with vehicle control (FIG. 1, panel C). Weight loss was still evident in the group injected with the m-calpain inhibitor compared to AOM only control group, but the mice quickly recovered their weight up to levels of AOM control group while mice injected with vehicle control exhibited continual low weights with little recovery. This indicates that the m-calpain inhibitor is protective but may be improved by changing the injection regimen. In the later stages of the protocol, the weight recovery for mice treated with the calpain-2 inhibitor reached the levels of mice with no DSS administration. Another sign of severe colitis for the DSS/AOM models is bloody diarrhea. Measurement of hemoglobin in the cecums by ELISA showed that mice treated with the calpain-2 inhibitor showed significantly lower levels of hemoglobin compared to vehicle control treated mice, and were reduced to levels found in the negative control group (FIG. 1, panel D).

### Example 2

### Treating Inflammation with a Calpain-2 Inhibitor

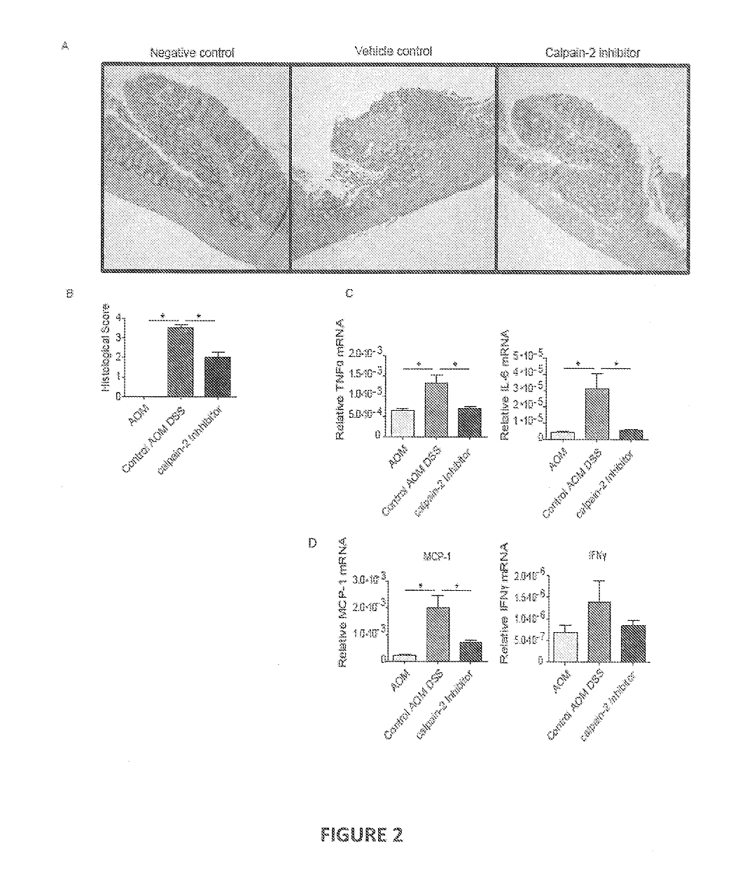

Histological evaluation of colon tissue in mice treated with the calpain-2 inhibitor showed a significant decrease in colitis pathology and inflammatory infiltration compared to vehicle control treated mice (FIG. 2, Panels A and B). Mice subjected to the AOM/DSS model were sacrificed on day 63 and colons removed and assessed by histology and cytokine mRNA analyses. The histology scores were not completely reduced to the levels of the negative control mice, which suggests the calpain-2 inhibitor was not able to completely block tissue damage caused by DSS treatment and instead inhibited the inflammatory response that contributed to pathology. Sections from vehicle control group exhibited severe colitis as indicated by a higher degree of cellular infiltration into the submucosa and muscularis propria (large arrows) and highly disrupted crypt architecture (small arrows) compared with WT and WT→WT controls, respectively. To further assess inflammation, mouse colon tissues were harvested at the end of the chronic colitis model and mRNA levels for different cytokines measured by real-time PCR. Results showed that calpain-2 inhibitor treatment lowered TNFα, IL-6, and MCP-1 mRNA levels compared to vehicle control treated mice and approached levels exhibited by the untreated control mice (FIG. 2, Panel D). IFNγ also appeared to be decreased but the levels were not significantly different compared to vehicle control treated mice.

### Example 3

### Inactivation of Inflammatory Macrophages with Calpain-2 Inhibitor

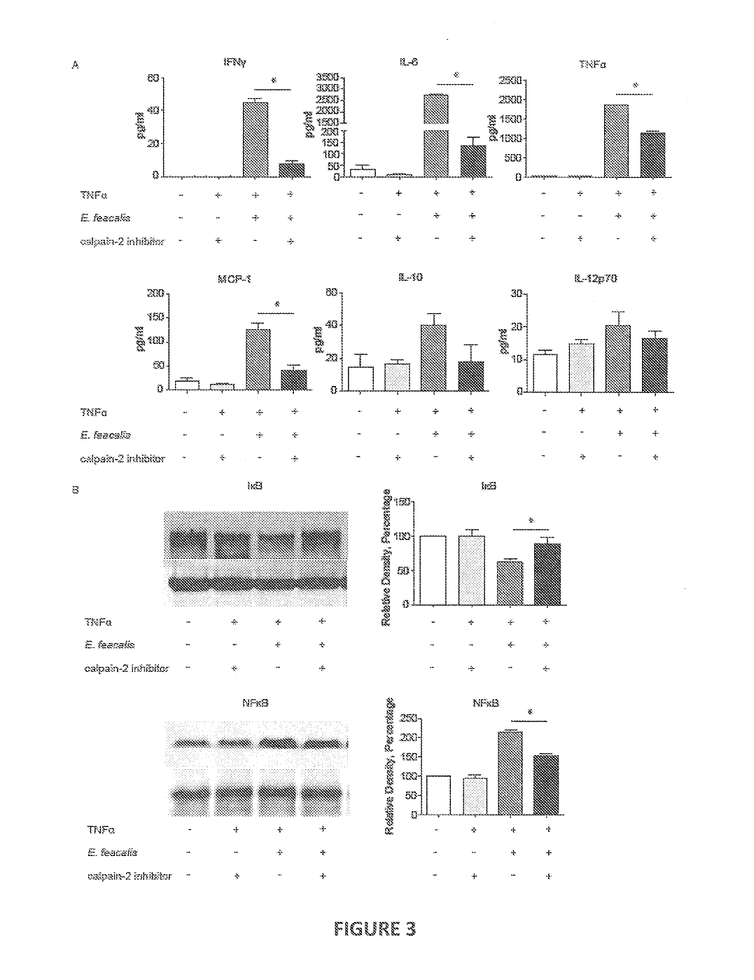

In macrophages, calpain cleaves IκB and releases NFκB that then translocates to the nucleus to activate transcription of inflammatory cytokine genes (Fenouille, N. et al. J. Pathol. 227, 118-129, 2012), and CAST was shown to be important for reducing this signaling event. To determine if a similar mechanism may be contributing to the effect of the calpain-2 inhibition in the DSS model of colitis, macrophages were analyzed for NFκB activation and inflammatory cytokine secretion. Bone marrow derived macrophages were primed 24 h with TNFα (20 ng/ml) and activated with E. faecalis (1 μg/ml) for 1 h in the presence or absence of calpain-2 inhibitor (20 μg/ml). Supernatants were evaluated for a panel of cytokines including IFNγ, TNFα, IL-6, and MCP-1, IL-10, and IL-12. Real-time PCR was performed on total RNA extracted from colon tissues to access levels of cytokines. Media was collected from wells and cytokines assessed by CBA kit (BD Biosciences). For IκB blotting cells were lysed and SDS-PAGE was performed. Membranes were stained with anti-IκB (Cell Signalling) and anti-βactin (Santa Cruz). For NfκB blotting an NFκB Activation Kit (Five Photon) was used to isolate nuclear fractions. These fractions were transferred using SDS-PAGE. The membranes were stained with anti-NFκB (Santa Cruz) or anti-TATA (Abcam). Data are mean±SE (n=3) and represent results from three independent experiments. *p<0.05; one way ANOVA followed by Tukey post-test. Results showed that calpain-2 inhibitor treatment during priming and activation led to a significant decrease in the production of the inflammatory cytokines IFNγ, TNFα, IL-6, and MCP-1, while IL-10 and IL-12 were not significantly affected (FIG. 3, Panel A). Importantly, those cytokines most affected by the calpain-2 are regulated by NFκB.

To analyze NFκB signaling, TNFα primed macrophages were activated with E. faecalis in the presence of the calpain-2 inhibitor or DMSO as a control. Levels of IκB and nuclear NFκB were analyzed by western blot. Results show that calpain-2 inhibition decreased 1 KB degradation and NFκB localization to the nucleus in activated macrophages (FIG. 3, Panel B).

### Example 4

### Treating Colorectal Cancer with Calpain-2 Inhibitor

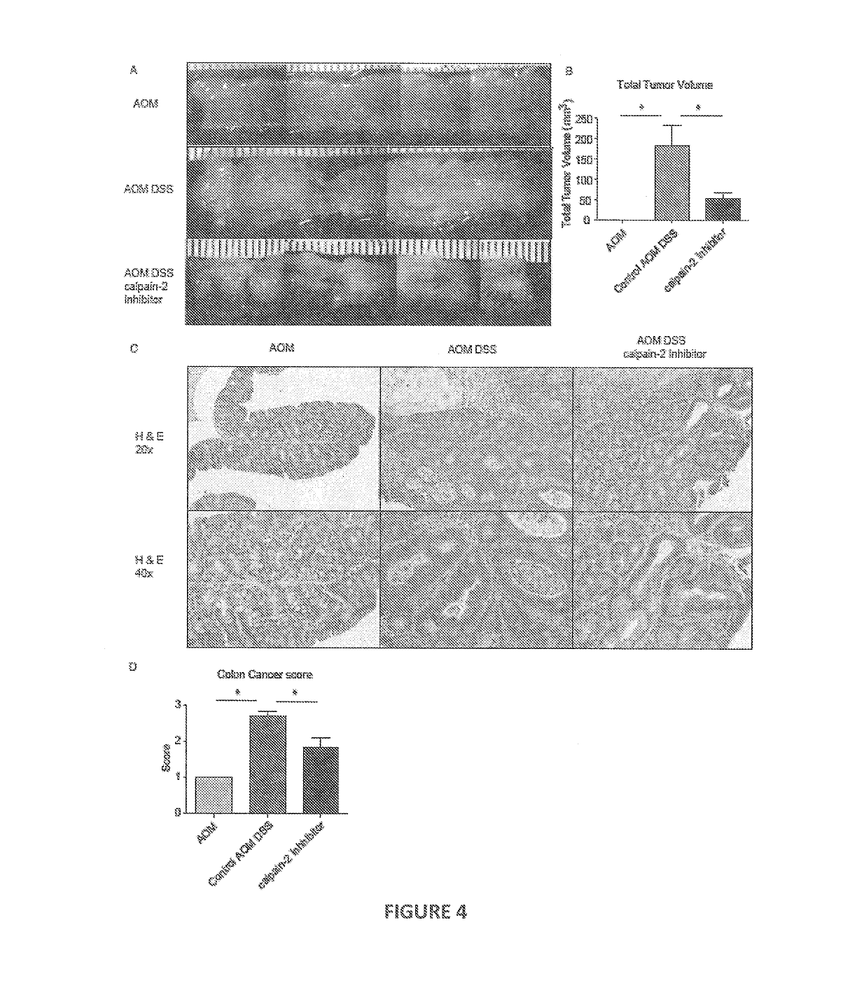

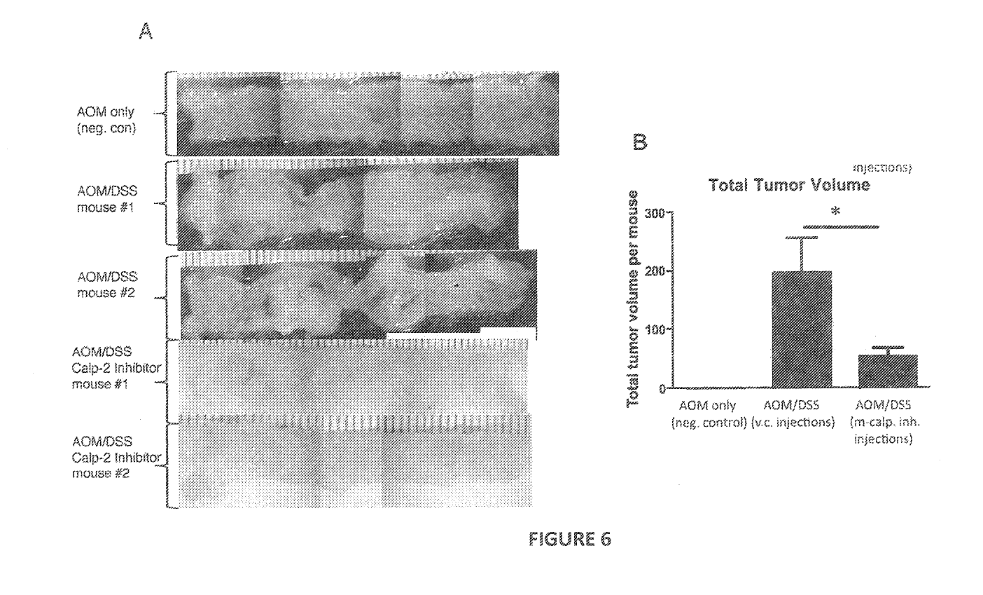

Colorectal cancer (“colitis associated cancer”, or “CAC”) was modelled using the outcome of the AOM/DSS model. The effects of intervention with the calpain-2 inhibitor on colorectal cancer formation/progression were determined. A cohort of 6 mice/group were included and the selective m-calpain inhibitor Z-LLY-CH2F was interperitoneally injected for 5 days followed by 2 days with no injections. This was started after the first signs of weight loss and blood in the feces on day 8, and the 5 days injection/2 days no injection schedule was continued through day 62. On day 63 of the AOM/DSS model the mice were sacrificed and the colons removed and imaged to determine tumor volumes. Mice treated with the calpain-2 inhibitor had significantly lower total tumor volume (reduced by 70%) compared to vehicle control treated mice (FIG. 4, Panels A and B; and FIG. 6, Panels A and B). For vehicle controls, large colon tumors are evident in the distal regions near the rectum (left side of FIG. 4, Panels A and B; and left side of FIG. 6, Panels A and B); calpain-2 inhibitor treatment significantly reduced the total side of these tumors. An average of 2 tumors/mouse were found in both the vehicle control and m-calpain treated groups, but the tumors were significantly smaller in those mice treated with the m-calpain inhibitor. A blinded histological assessment of the colons was performed using a 4-point system as described in the Methods section. The calpain-2 inhibitor treated mice showed significantly lower CAC pathology compared to vehicle control treated mice, although those mice treated with the inhibitor were not completely free of cancer (FIG. 4, Panels C-D). For example, the mice treated with the calpain-2 inhibitor had less generalized tissue disruption, pleiomorphism, and hyperchromasia. Overall, intervention with the calpain-2 inhibitor reduced the development of CAC as assessed by macroscopic analyses and histology.

### Example 5

### Treating Colon Cancer Cell Proliferation with Calpain-2 Inhibitor

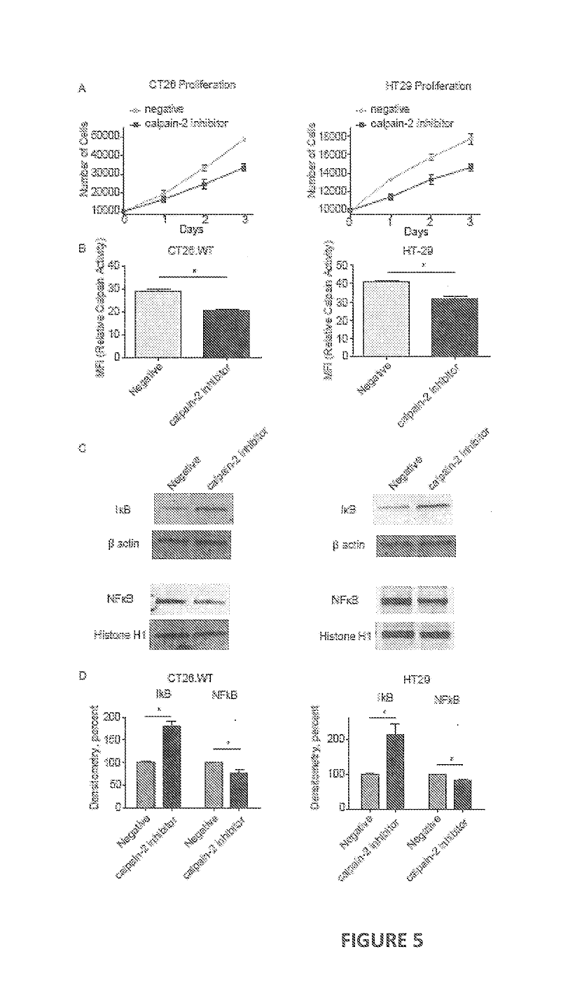

The effect of the m-calpain inhibitor on CAC was surprising and could be attributed to reduced inflammation and/or direct inhibition of colorectal tumor progression. The fact that m-calpain expression is upregulated in CAC and other cancers indicates that m-calpain functions to promote the progression of the tumors (Leloup, L. & Wells, A. Expert Opinion on Therapeutic Targets 15, 309-323, 2011; Liu, T., Mendes, D. E. & Berkman, C. E. Int. J. Oncol. 44, 467-472, 2014). It has also been demonstrated that calpain activity can lead to more aggressive and chemotherapy resistant cancers (Fenouille, N. et al. J. Pathol. 227, 118-129, 2012; Storr, S. J. et al. J. Cell. Mol. Med. 16, 2422-2428, 2012). In order to determine if the calpain-2 inhibitor had any direct effects on CAC, mouse CT26.WT and human HT-29 colorectal cancer cell lines were cultured in the presence of the m-calpain inhibitor. CT-26 WT mouse colon cancer cells and HT-29 human colorectal cancer cells were plated at a concentration of 1×10⁴ cells per well in a 96-well plate with either m-calpain inhibitor or DMSO. M-Calpain activity in both cell lines was reduced and this corresponded with a reduction in proliferation in both of these cell lines (FIG. 5, Panels A-B). Media was replaced every 2 days with fresh calpain-2 inhibitor (20 μg/ml) or DMSO. The number of cells was determined each day by MTS reagent (Promega). Cells were also plated in 6-well plates at a concentration of 2×10⁶ cells per well. Cells were serum starved in 0.2% serum RPMI in the presence of m-calpain inhibitor or DMSO for 1 hour. Calpain activity inhibition in the cell lines was determined using a Calpain Activity Assay Kit (BioVision). Cells were also plated in 6-well plates at a concentration of 2×10⁶ cells per well. Cells were serum starved in 0.2% serum RPMI in the presence of calpain-2 inhibitor or DMSO for 1 hour. FIG. 5, panel C shows western blots of cell lysate for IκB blots and on density centrifuged nuclear samples for NFκB blots. The blots were examined by densitometry. As shown above, treating activated macrophages with the m-calpain inhibitor reduced IκB degradation and NFκB nuclear localization. Western blot analyses were performed on colorectal cancer cell lines treated with m-calpain inhibitor to determine IκB levels in lysates and NFκB in the nuclear fractions. M-Calpain inhibition reduced IκB degradation and NFκB nuclear localization in the colorectal cancer cell lines (FIG. 5, Panels C-D). These results together with the results from the macrophage experiments suggest that the 70% reduction in colon tumors from the m-calpain inhibitor is likely due to both decreased inflammation as well as direct effects on tumor cell proliferation.

The m-calpain inhibitor reduces NFkB activation in both macrophages and colon cancer cells. This leads to reduced secretion of pro-inflammatory cytokines by macrophages which drive tumor progression, and also directly reduces colon tumor cell proliferation. Other m-calpain regulated pathways in both macrophages and colon cancer cells are inhibited by the m-calpain inhibitor, including phosphorylation of Akt. In macrophages, a major effect on FAK cleavage is demonstrated with the m-calpain inhibitor, while this effect is not apparent in colon cancer cells. Altogether, this data indicates that m-calpain inhibition reduces both inflammation during macrophage-driven colitis which reduces pro-inflammatory growth of colon cancer cells. Additionally, m-calpain inhibition in the cancer cells reduces proliferation of said cells.

### Example 6

### Delivery of Synthetic m-Calpain Inhibitors to the Colon

Encapsulation of zLLY-CH2F in Hydrogel NP.

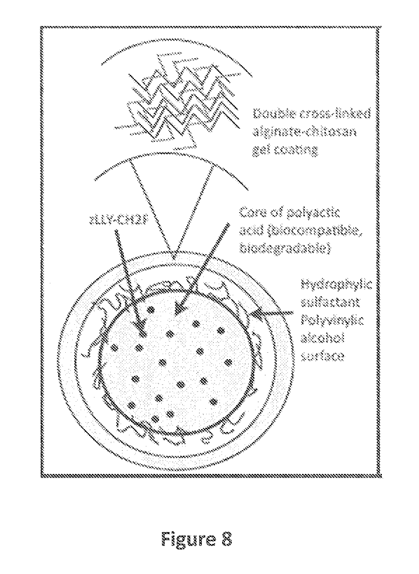

An exemplary specific, synthetic m-calpain inhibitor of this invention is formulated in nanoparticle formulation for delayed delivery to the colon. Construction and loading of 400 nm diameter nanoparticles (NP) coated by chitosan and alginate is performed. (See Laroui H, Dalmasso G, Nguyen H T, et al. Gastroenterology 138: 843-853 e841-842, 2010). In brief, double-emulsion/solvent evaporation (water in oil in water) are performed with zLLY-CH2F loaded into polylactic acid NP during the first emulsion of the synthesis process, which is then recovered in polyvinylic alcohol (FIG. 8). To measure zLLY-CH2F loaded onto NP, bovinse serum albumin (BSA) concentration is measured by uv spectroscopy in the final washing solutions. The encapsulation rate of BSA is determined based on the initial BSA concentration. After each final washing to remove extra polyvinyl alcohol by centrifugation of NP suspension, the supernatant is collected. The accumulated washing volumes are used to determine the concentration of BSA present in the supernatant, thus providing the mass of protein not encapsulated. A mass balance is performed to determine the amount of BSA that is loaded into the NP according to the known initial concentration. The nanoparticles are then encapsulated by a hydrogel solution prepared from alginate and chitosan solutions mixed at a 1:1 ratio for a final concentration of 7 and 3 g/L, respectively, and homogenized for 24 h. The size distribution of NPs (mean diameter and standard deviation) are determined by photon correlation spectroscopy. The final suspension is freeze-dried and later resuspended in DMEM cell media and sonicated prior to use.

### In Vitro Testing of NP Toxicity and Effectiveness.

A model of colorectal cancer cells used are Caco2-BBE enterocyte cells, which are a continuous cell line of heterogeneous human epithelial colorectal adenocarcinoma cells (J. Fogh, G. Trempe, “Human Tumor Cells In Vitro” (J. Fogh, ed.), Plenum, 1975, pages 115-141). Caco2-BBE enterocyte cells (5×10⁴) are plated in 96-well plates in DMEM with 5% FBS overnight at 70% confluency. The NP and vehicle control NP are added to the cells in increasing concentrations for 24, 48, and 72 h. Cell viability are assayed by Vybrant MTT Cell Viability kit (Life Technologies) and absorbance read on a Molecular Devices plate reader. The doses of the calpain-inhibiting compound tested range from about 0.1 ng/kg to 4.0 mg/kg, from about 0.5 ng/kg to about 0.5 mg/kg, from about 1.0 ng to about 100 ug/kg, from about 10 ng/kg to about 10 ug/kg, from about 100 ng/kg to about 1 ug/kg, from about 1 mg/kg to about 100 mg/kg, and from about 10 mg/kg to about 1000 mg/kg. The dosages are then tested for inhibiting calpain activity using the Calpain activity assay kit (BioVision, Inc.). As controls, the unencapsulated calpain-2 and calpain-1 inhibitors are added at established concentrations of 20 μg/ml to distinguish which isoform is affected by the NP (see Huang Z, Hoffmann F W, Norton R L, et al. The Journal of biological chemistry 286: 34830-34838, 2011). The range of doses tested are effective in Caco2-BBE cells and are then tested on primary peritoneal mouse macrophages to confirm nontoxicity and effective inhibition of calpain-2 activity. The nontoxic dosage that reduces calpain activity to those equivalent to unencapsulated zLLY-CH2F is used in in vivo studies.

### In Vivo Testing of NP

The zLLY-CH2F loaded nanoparticles are delivered by oral gavage to mice daily for 5 days followed by 2 days rest. Mice tolerate delivery volumes up to 10 mL/kg, and mice also tolerate long-term gavage on this schedule. Control mice receive vehicle control NP by gavage. On day 8, mice are sacrificed and the following tissues are harvested: colon, small intestine, spleen, heart, liver, kidney, brain, and lung. Tissues are stored at −80° C. Thawed tissues are analyzed for calpain activity as previously described (Huang Z, Rose A H, Hoffmann F W, et al. J Immunol; 191: 3778-3788, 2013; Huang Z, Hoffmann F W, Norton R L, et al. The Journal of biological chemistry; 286: 34830-34838, 2011). Inhibition of total calpain activity in each tissue is compared in mice treated with zLLY-CH2F loaded NP versus vehicle control NP. To determine the relative inhibition of each calpain isoform (calpain-1 and -2), each tissue lysate includes total calpain measurement with unencapsulated calpain-2 or calpain-1 inhibitor added at 20 μg/ml or DMSO as a control.

### Therapeutic Efficacy of NP in AOM/DSS Model.

The in vivo dosage of NP is used as intervention beginning on day 8 similar to the unencapsulated zLLY-CH2F as described above. The gavage of mice is performed on a schedule of 5 day gavage with 2 day rest through the remainder of the 63 day protocol. All of the measurements of colitis and CAC are the same as described above. Vehicle Control mice receive vehicle control loaded NP and Negative Control mice only receive AOM injection with no DSS and no NP. All groups contain 20 mice/group as this provides the power required for discriminating effects of intervention as shown above.

### Results

The optimal dosage of zLLY-CH2F loaded nanoparticles that reduces both colitis and CAC to levels that are equivalent to interperitoneal injected unencapsulated zLLY-CH2F is determined to be from about 0.1 ng/kg to 4.0 mg/kg, from about 0.5 ng/kg to about 0.5 mg/kg, from about 1.0 ng to about 100 ug/kg, from about 10 ng/kg to about 10 ug/kg, from about 100 ng/kg to about 1 ug/kg, from about 1 mg/kg to about 100 mg/kg, and from about 10 mg/kg to about 1000 mg/kg. The composition of the NP polymer results in preferential decreased calpain-2 activity in the colon compared to other tissues examined. The activity of the calpain-2 activity in the colon for calpain-2 NP-treated mice is reduced by about 50%, or about 55%, or about 60%, or about 65%, or about 70%, or about 75%, or about 80%, or about 85%, or about 90%, or about 95%, or about 97.5%, or about 99%, or about 100% compared to calpain-2 activity in other tissues tested. The therapeutic effect of zLLY-CH2F loaded NP is determined to result in a reduction of tumor volume of the calpain inhibitor NP treated mice compared to vehicle control of about 65%, about 70%, about 75%, about 80%, about 85%, about 90%, about 95%, about 97.5%, about 99%, or about 100%. The expression of the inflammatory cytokines IFNγ, TNFα, IL-6, and MCP-1 in the colon for calpain-2 NP-treated mice is reduced compared to other tissues tested. The results indicate that synthetic m-calpain inhibitors can be targeted to the colon in a manner that reduces off-tissue effects while maintaining a strong therapeutic inhibition of colitis and/or colorectal cancer.

What is claimed is:

1. A method of treating colorectal cancer or inflammatory bowel disease, comprising administering to a subject in need thereof, a therapeutically effective amount of a pharmaceutical composition comprising a m-calpain selective inhibitor, or a pharmaceutically acceptable salt, solute, or hydrate thereof.

2. The method of claim 1, wherein the inflammatory bowel disease is ulcerative colitis, Crohn's disease, collagenous colitis, lymphocytic colitis, ischaemic colitis, diversion colitis, Behçet's syndrome, infective colitis or indeterminate colitis.

3. The method of claim 1, further comprising administering a therapeutically effective amount of an agent useful for treating inflammatory bowel disease.

4. The method of claim 3 comprising administering sequentially the m-calpain selective inhibitor, or a pharmaceutically acceptable salt, solute, or hydrate thereof, and a therapeutically effective amount of an agent useful for treating inflammatory bowel disease.

5. The method of claim 3, wherein the m-calpain selective inhibitor, or a pharmaceutically acceptable salt thereof, and the therapeutically effective amount of an agent useful for treating inflammatory bowel disease are administered within about one hour of each other, within about one day or each other, or within about one week of each other, or within about one month of each other, and optionally, the m-calpain selective inhibitor is administered first.

6. The method of claim 3, wherein the therapeutic agent useful for treating inflammatory bowel disease is selected from one of the following classes of compounds: 5-aminosalicyclic acids, corticosteroids, thiopurines, tumor necrosis factor-alpha blockers and JAK inhibitors.

7. The method of claim 6, wherein the therapeutic agent useful for treating inflammatory bowel disease is selected from one or more of the following agents: Prednisone, Humira, Lialda, Imuran, Sulfasalazine, Pentasa, Mercaptopurine, Azathioprine, Apriso, Simponi, Enbrel, Humira Crohn's Disease Starter Pack, Colazal, Budesonide, Azulfidine, Purinethol, Proctosol HC, Sulfazine EC, Delzicol, Balsalazide, Hydrocortisone acetate, Mesalamine, Proctozone-HC, Sulfazine, Orapred ODT, Mesalamine, Azasan, Asacol HD, Dipentum, Prednisone Intensol, Anusol-HC, Rowasa, Azulfidine EN-tabs, Veripred 20, Uceris, Adalimumab, Hydrocortisone, Colocort, Pediapred, Millipred, Azathioprine injection, Prednisolone sodium phosphate, Flo-Pred, Aminosalicylic acid, ProctoCream-HC, 5-aminosalicylic acid, Millipred DP, Golimumab, Prednisolone acetate, Rayos, Proctocort, Paser, Olsalazine, Procto-Pak, Purixan, Cortenema, Giazo, Vedolizumab, Entyvio, Micheliolide, and Parthenolide.

8. The method of claim 1, whereby treating colorectal cancer is indicated by the inhibition or reduction of cancer progression.

9. The method of claim 1, further comprising administering a therapeutically effective amount of an agent useful for treating colorectal cancer.

10. The method of claim 9 comprising administering sequentially the m-calpain selective inhibitor, or a pharmaceutically acceptable salt, solute, or hydrate thereof, and the therapeutically effective amount of an agent useful for treating colorectal cancer.

11. The method of claim 9, wherein the m-calpain selective inhibitor, or a pharmaceutically acceptable salt thereof, and the therapeutically effective amount of an agent useful for treating colorectal cancer are administered within about one hour of each other, within about one day or each other, or within about one week of each other, or within about one month of each other; and optionally, the m-calpain selective inhibitor is administered first.

12. The method of claim 9, wherein the therapeutic agent useful for treating colorectal cancer is selected from one of the following: Adrucil (Fluorouracil), Aclarubicin, Avastin (Bevacizumab), Betaseron (interferon beta-1b), BIBF-1120 (3-Z-[I-(4-(N-((4-methyl-piperazin-1-yl)-methylcarbonyl)-N-methyl-amino)-anilino)-I-phenyl-methylene]-6-methoxycarbonyl-2-indolinone), BIBW 2992 (3-Z-[I-(4-(N-((4-methyl-piperazin-1-yl)-methylcarbonyl)-N-methyl-amino)-anilino)-I-phenyl-methylene]-6-methoxycarbonyl-2-indolinone), Adriamycin, Daunomycin, Aclarubicin, Amrubicin, Idarubicin, Epirubicin, Pirarubicin, Dacarbazine, Mitoxantrone Bevacizumab, Camptosar (Irinotecan Hydrochloride), Capecitabine (Xeloda), Cisplatin, Carboplatin, Satraplatin, analogues of Cisplatin, Efudex (Fluorouracil), Eloxatin (Oxaliplatin), Erbitux (Cetuximab), Fluorouracil, Irinotecan Hydrochloride, Leucovorin Calcium, Oxaliplatin, Panitumumab, Regorafenib, Stivarga (Regorafenib), Vectibix (Panitumumab), Wellcovorin (Leucovorin Calcium), Zaltrap (Ziv-Aflibercept), CAPDX, FOLFIRI, FOLFIRI-BEVACIZUMAB, FOLFIRI-CETUXIMAB, FOLFOX, FU-LV, and XELOX.

13. The method of claim 1 wherein the effective amount of the compound ranges from about, 0.01 mg/kg to about 1 mg/kg, from about 0.02 mg/kg to about 50 mg/kg, from about 0.1 mg/kg to 50 mg/kg, from about 0.1 mg/kg to 5 mg/kg, from about 0.1 mg/kg to 2 mg/kg, or from about 0.1 mg/kg to 1 mg/kg.

14. The method of claim 1 wherein the effective amount comprises one or more effective doses of the compound which are administered orally, intradermal, intramuscularly, anally, intraperitoneally, subcutaneously, intravenously, intramuscularly, or via epidural.

15. The method of claim 1 wherein the effective amount comprises one or more doses of a therapeutically effective amount of the compound which are encapsulated in a particle.

16. The method of claim 15, wherein the particle is a particle selected from one of the following: polylactide (PLA) nanoparticles, poly-DL-lactic acid (PDLLA) microspheres, poly (lactic acid) nanoparticles, chitosan-modified poly (D,L-lactide-co-glycolide) nanospheres (CS-PLGA NSs), chitosan-alginate coated nanoparticle, solid lipid nanoparticles (SLNs), grapefruit-derived nanoparticles (GDNs), silicon nanoparticles, polylactic-co-glycolic acid (PLGA) nanoparticles, pH-sensitive Eudragit P-4135F nanoparticles, thioketal nanoparticles (TKNs) made from the polymer poly-PPADT (1,4-phenyleneacetone dimethylene thioketal), lipopolysaccharides (LPS), and type B gelatin enclosed in poly(e-caprolactone) (PCL) microspheres.

17. The method of claim 15, wherein the therapeutically effective amount of compound is from about 0.1 ng/kg to 4.0 mg/kg, from about 0.5 ng/kg to about 0.5 mg/kg, from about 1.0 ng to about 100 ug/kg, from about 10 ng/kg to about 10 ug/kg, from about 100 ng/kg to about 1 ug/kg, from about 1 mg/kg to about 100 mg/kg, or from about 10 mg/kg to about 1000 mg/kg.

18. The method of claim 1, wherein the selective m-calpain inhibitor is a modified peptide that comprises at least one partial leucine moiety and an alkyl halide group.

19. The method of claim 1, wherein the modified peptide is Calpain Inhibitor IV.

20. The method of claim 1, wherein the m-calpain selective inhibitor is an irreversible inhibitor of m-calpain.

21. The method of claim 1, wherein the m-calpain selective inhibitor irreversibly inhibits m-calpain with a k₂ rate constant greater than about 28,000 M⁻¹s⁻¹, or greater than 25,000 M⁻¹s⁻¹, or greater than 20,000 M⁻¹s⁻¹, or greater than 15,000 M⁻¹s⁻¹, or greater than 10,000 M⁻¹s⁻¹ or greater than 5,000 M M⁻¹s⁻¹.

22. The method of claim 1, wherein the m-calpain selective inhibitor is a reversible inhibitor of m-calpain.

23. The method of claim 1, wherein the m-calpain selective inhibitor reversibly inhibits m-calpain with a IC₅₀ of less than about 100 nM, or less than 50 nM, or less than 40 nM, or less than 30 nM, or less than 20 nM, or less than 10 nM, or less than 1 nM.

24. The method of claim 1, wherein the m-calpain selective inhibitor inhibits or binds to m-calpain more than μ-calpain.

25. The method of claim 1, wherein the m-calpain selective inhibitor is formulated to preferentially release in the colon.

26. A method of inhibiting the growth of tumor cells or a colony of tumor cells comprising contacting said tumor cells with an effective amount of an selective inhibitor of m-calpain.

27. A kit, comprising a first composition comprising an effective amount of a selective m-calpain inhibitor wherein the selective m-calpain inhibitor is Calpain Inhibitor IV, or a pharmaceutically acceptable salt, solute, or hydrate thereof; and a second composition a therapeutic agent useful for treating inflammatory bowel disease or colorectal cancer, or a pharmaceutically acceptable salt, solute, or hydrate thereof, together with instructions for administering the first composition and the second composition to a patient suffering from colitis or inflammatory bowel disease or colorectal cancer.

28. The kit of claim 27, wherein the first and second composition are administered in combination, are administered simultaneously, are administered separately, are administered sequentially, or are administered in a controlled manner.
